(19) United States
(12) Patent Application Publication
(10) Pub. No.: US 2003/0211078 A1
(43) Pub. Date: Nov. 13, 2003

(54) Pseudo-antibody constructs

(76) Inventor: George A. Heavner, Malvern, PA (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/309,722

(22) Filed: Dec. 4, 2002

Related U.S. Application Data
(60) Provisional application No. 60/336,707, filed on Dec. 7, 2001.

(51) Int. Cl. A61K 38/19; A61K 38/17; A61K 31/715; C07K 16/46; C07K 14/52; A61K 39/395

(52) U.S. Cl. 424/85.1; 424/130.1; 514/12; 514/54; 530/351; 530/350; 530/387.1; 536/123; 525/54.1

(57) ABSTRACT

This invention relates to novel pharmaceutically useful compositions that bind to a biological molecule, having improved circulatory half-life, increased avidity, increased affinity, or multifunctionality, and methods of use thereof. The present invention provides a pseudo-antibody comprising an organic moiety covalenty coupled to at least two target-binding moieties, wherein the target-binding moieties are selected from the group consisting of a protein, a peptide, a peptidomimetic, and a non-peptide molecule that binds to a specific targeted biological molecule. The pseudo-antibody of the present invention may affect a specific ligand in vitro, in situ and/or in vivo. The pseudo-antibodies of the present invention can be used to measure or effect in an cell, tissue, organ or animal (including humans), to diagnose, monitor, modulate, treat, alleviate, help prevent the incidence of, or reduce the symptoms of, at least one condition.

This application claims priority to U.S. provisional application 60/336,707, filed Dec. 7, 2001, and which application is entirely incorporated herein by reference.

### FIELD OF THE INVENTION

This invention relates to novel pharmaceutically useful compositions that bind to a biological molecule, having improved circulatory half-life, increased avidity, increased affinity, or multifunctionality, and methods of use thereof.

### BACKGROUND OF THE INVENTION

Numerous pharmaceutical compounds and peptides have been identified that bind to a biological molecule and that affect biological activity. Recombinant protein technology has provided numerous promising therapeutic agents. Advances in protein formulation and chemical modification of these therapeutic proteins have lead to improved resistance to proteolytic enzymes and decreased immunogenicity, thus increasing the therapeutic protein's stability, circulatory half-life, and biological activity.

Antibodies provide an example of recombinant proteins with great therapeutic potential. Full antibodies are bivalent molecules composed of two identical Fab domains and an Fc domain. The Fab domains contain two identical binding sites, sometimes referred to as paratopes, each within the variable regions at the N-termini of the Fab domains, and comprised of complementarity determining regions (CDRs). Antibodies have additional functionality in their Fc domains, that can offer additional functionality beyond the binding of the CDRs in the variable regions. There are instances, however, when Fc-mediated activity can be disadvantageous. For example, an antibody fragment that binds to the GPIIb/IIIa receptors on platelets can block platelet aggregation, but the presence of an Fc domain would result in platelet clearance and thrombocytopenia. Antibodies can be subjected to proteolysis to remove the Fc domain, creating either Fab or Fab′₂ fragments. These non-glycosylated antibody fragments have molecular weights of approximately 50,000 and 100,000 where the parent antibodies have molecular weights of approximately 150,000 and can be glycosylated. And although antibody fragments may be advantageous therapeutically, antibody fragments are generally cleared at a faster rate than the intact antibodies. Capon et al., 337 NATURE 525-31 (1989).

A limited number of constructs have been prepared where the Fab domains have been modified. In particular, synthetic moieties such as PEG have been added to the Fab to increase the molecular weight and slow down clearance. See, e.g., WO 00/26256; published May 11, 2000.

Antibodies, proteins, and peptides have been modified with polyethyleneglycol (PEG) to increase half-life, decrease degradation and decrease immunogenicity. Derivatized PEG compounds have been discussed previously. See U.S. Pat. No. 5,438,040.

Yet, there remains a need in the field for improved modified therapeutic antibodies. More specifically, these modifications, as described herein, improve the pharmacokinetic properties (e.g., increase in vivo serum half-life) without significantly affecting the antigen-binding properties (e.g., affinity) of the antigen-binding moieties, while potentially increasing avidity and providing, for example, a single pseudo-antibody that binds more than one type of antigen or receptor. This invention thus provides for the construction of entirely new families of pseudo-antibodies (Ψ Abs) using either Fab or Fab′ fragments prepared from antibodies, single chain antibodies (sFᵥ), peptides that bind to proteins or other biological molecules, or organic compounds that bind to proteins or other biological molecules.

### SUMMARY OF INVENTION

The present invention provides a pseudo-antibody comprising an organic moiety covalenty coupled to two or more identical target-binding moieties, wherein said target-binding moieties are selected from the group consisting of a protein, a peptide, a peptidomimetic, and a non-peptide molecule that binds to a specific targeted biological molecule. The present invention also provides for a pseudo-antibody comprising an organic moiety covalenty coupled to two or more different target-binding moieties, wherein said target-binding moieties are selected from the group consisting of a protein, a peptide, a peptidomimetic, and a non-peptide molecule that binds to a specific targeted biological molecule.

The pseudo-antibody of the present invention may affect a specific ligand, such as where the pseudo-antibody modulates, decreases, increases, antagonizes, angonizes, mitigates, alleviates, blocks, inhibits, abrogates and/or interferes with at least one biological molecule's activity or binding, or with a receptor activity or binding, in vitro, in situ and/or in vivo. The pseudo-antibodies of the present invention can be used to measure or effect in an cell, tissue, organ or animal (including humans), to diagnose, monitor, modulate, treat, alleviate, help prevent the incidence of, or reduce the symptoms of, at least one condition. The pseudo-antibody constructs may be used to treat stenosis and/or restenosis following a vascular intervention, to prevent ischemia, to inhibit the growth and/or metastasis of a tumor, to inhibit a biological process mediated by the binding of a ligand to either or both of GPIIb/IIIa and αᵥβ₃, expressed on the plasma membrane of a cell, or to inhibit angiogenesis. Such a method can comprise administering an effective amount of a composition or a pharmaceutical composition comprising at least one pseudo-antibody to a cell, tissue, organ, animal or patient in need of such modulation, treatment, alleviation, prevention, or reduction in symptoms, effects or mechanisms. The effective amount can comprise an amount effective amount per single, multiple or continuous administration.

### BRIEF DESCRIPTION OF THE DRAWINGS

FIG. 1 depicts a comparison of the inhibition of platelet aggregation by two pseudo-antibodies (7E3 Fab′ (PEG_{3.4K}-DSPE)₂ and 7E3 Fab′ (PEG_{3.4K}-PAL)₂) and one unmodified antibody fragment (7E3 Fab).

FIG. 2 depicts a comparison of the inhibition of platelet aggregation by two pseudo-antibodies (7E3 Fab′ (PEG_{5K})₂ and 7E3 Fab′ (PEG_{10K})₂) and one unmodified antibody fragment (ReoPro®).

FIG. 3 depicts a comparison of in vivo circulating half-life, in mice, of two pseudo-antibodies, 7E3 Fab′ (PEG_{3.4K}-DSPE)₂ and 7E3 Fab′ (PEG_{5K})₂.

### DETAILED DESCRIPTION

It is to be understood that this invention is not limited to the particular methodology, protocols, constructs, formulae and reagents described and as such may vary. It is also to be understood that the terminology used herein is for the purpose of describing particular embodiments only, and is not intended to limit the scope of the present.

It must be noted that as used herein and in the appended claims, the singular forms “a,” “and,” and “the” include plural reference unless the context clearly dictates otherwise. Thus, for example, reference to “a gene” is a reference to one or more genes and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are incorporated herein by reference for the purpose of describing and disclosing, for example, the constructs and methodologies that are described in the publications which might be used in connection with the presently described invention. The publications discussed above and throughout the text are provided solely for their disclosure prior to the filing date of the present application. Nothing herein is to be construed as an admission that the inventor is not entitled to antedate such disclosure by virtue of prior invention.

The present invention provides for entirely new families of pseudo-antibodies (Ψ Abs) using peptides that bind to antigens, receptors, proteins or other biological molecules, either Fab or Fab′ fragments prepared from antibodies, single chain antibodies (sFᵥ), or organic compounds that bind to proteins or other biological molecules (target-binding moieties). The target-binding moieties may be peptides identified or produced by various methods known in the art. The method of obtaining these moieties, or the physical characteristics of these moieties, are not limitations of the invention. Preferred structures are those that bind to a biological molecule to block binding to another biological molecule or bind to a biological molecule to initiate a biological event. Some advantages of the invention described herein are that it presents molecules that bind to biomolecules and: (a) enhances their avidity (the functional combining strength of an target-binding moiety with its target, which is related to both the affinity of the reaction between the epitopes and the paratopes, and the valencies of the target-binding moiety and target); (b) provides multivalent constructs; (c) increases their circulating half-lives by increasing molecular size; (d) creates specific binding to multiple compounds by a single molecule; and/or (e) allows the incorporation of lipids, fatty acids, carbohydrates, steroids, etc.; that can bind to molecules other than the primary biological molecules and affect distribution to specific locations (e.g., fatty acid adducts could bind to serum albumin to keep molecules in circulation or lipid adducts could be used to provide non-covalent attachment of constructs to lipid-coated stents).

The target-binding moiety of the pseudo-antibody may include an immunoglobulin, an integrin, an antigen, a growth factor, a cell cycle protein, a cytokine, a hormone, a neurotransmitter, a receptor or fusion protein thereof, a blood protein, an antimicrobial, or any fragment, or structural or functional analog thereof. In addition, the target itself may be an immunoglobulin, an integrin, an antigen, a growth factor, a cell cycle protein, a cytokine, a hormone, a neurotransmitter, a receptor or fusion protein thereof, a blood protein, an antimicrobial, or any fragment, or structural or functional analog thereof.

For example, in one embodiment of the invention, the target-binding moieties of the pseudo-antibody may be derived from human or non-human polyclonal or monoclonal antibodies. Specifically, these antibodies (immunoglobulins) may be isolated, recombinant and/or synthetic human, primate, rodent, mammalian, chimeric, humanized or CDR-grafted, antibodies and anti-idiotype antibodies thereto. Such moieties can be produced by enzymatic cleavage, synthetic or recombinant techniques, as known in the art and/or as described herein. Additionally, these binding moieties can also be produced in a variety of truncated forms in which various portions of antibodies are joined together chemically by conventional techniques, or prepared as a contiguous protein using genetic engineering techniques. As used presently, an “antibody,” “antibody fragment,” “antibody variant,” “Fab,” and the like, include any protein- or peptide-containing molecule that comprises at least a portion of an immunoglobulin molecule, such as but not limited to at least one CDR of a heavy or light chain or a ligand binding portion thereof, a heavy chain or light chain variable region, a heavy chain or light chain constant region, a framework region, or any portion thereof, or at least one portion of a receptor or binding protein, which can be incorporated into a pseudo-antibody of the present invention. Such antibody optionally further affects a specific ligand, such as but not limited to, where such antibody modulates, decreases, increases, antagonizes, agonizes, mitigates, alleviates, blocks, inhibits, abrogates and/or interferes with at least one target activity or binding, or with receptor activity or binding, in vitro, in situ and/or in vivo.

In one embodiment of the invention, such antibodies, or functional equivalents thereof, may be “human,” such that they are substantially non-immunogenic in humans. These antibodies may be prepared through any of the methodologies described herein, including the use of transgenic animals, genetically engineered to express human antibody genes. For example, immunized transgenic mice (xenomice) that express either fully human antibodies, or human variable regions have been described. WO 96/34096, published Oct. 31, 1996. In the case of xenomice, the antibodies produced include fully human antibodies and can be obtained from the animal directly (e.g., from serum), or from immortalized B-cells derived from the animal, or from the genes encoding the immunoglobulins with human variable regions can be recovered and expressed to obtain the antibodies directly or modified to obtain analogs of antibodies such as, for example, Fab or single chain Fv molecules. Id.

The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. The present invention thus encompasses antibody fragments capable of binding to a biological molecule (such as an antigen or receptor) or portions thereof, including but not limited to Fab (e.g., by papain digestion), Fab′ (e.g., by pepsin digestion and partial reduction) and F(ab′)₂ (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments. See, e.g., CURRENT PROTOCOLS IN IMMUNOLOGY, (Colligan et al., eds., John Wiley & Sons, Inc., NY, 1994-2001).

As with antibodies, other peptide moieties that bind a particular target protein or other biological molecule (target-binding peptides) are encompassed by the pseudo-antibody disclosed herein. Such target-binding peptides may be isolated from tissues and purified to homogeneity, or isolated from cells which contain the target-binding protein, and purified to homogeneity. Once isolated and purified, such target-binding peptides may be sequenced by well-known methods. From these amino acid sequences, DNA probes may be produced and used to obtain mRNA, from which cDNA can be made and cloned by known methods. Other well-known methods for producing cDNA are known in the art and may effectively be used. In general, any target-binding peptide can be isolated from any cell or tissue expressing such proteins using a cDNA probe such as the probe described above, isolating mRNA and transcribing the mRNA into cDNA. Thereafter, the protein can be produced by inserting the cDNA into an expression vector, such as a virus, plasmid, cosmid, or other vector, inserting the expression vector into a cell, proliferating the resulting cells, and isolating the expressed target-binding protein from the medium or from cell extract as described above. Alternatively, target-binding peptides may be chemically synthesized using the sequence described above and an amino acid synthesizer, or manual synthesis using chemical conditions well known to form peptide bonds between selected amino acids. Analogues and fragments of target-binding proteins may be produced by chemically modification or by genetic engineering. These fragments and analogues may then be tested for target-binding activity using known methods. See, e.g., U.S. Pat. No. 5,808,029 to Brockhaus et al., issued Sept. 15, 1998.

Alternatively, target-binding peptides, including antibodies, may be identified using various library screening techniques. For example, peptide library screening takes advantage of the fact that molecules of only “peptide” length (2 to 40 amino acids) can bind to the receptor protein of a given large protein ligand. Such peptides may mimic the bioactivity of the large protein ligand (“peptide agonists”) or, through competitive binding, inhibit the bioactivity of the large protein ligand (“peptide antagonists”). Phage display peptide libraries have emerged as a powerful method in identifying such peptide agonists and antagonists. In such libraries, random peptide sequences are displayed by fusion with coat proteins of filamentous phage. Typically, the displayed peptides are affinity-eluted against an immobilized extracellular domain of an antigen or receptor. The retained phages may be enriched by successive rounds of affinity purification and repropagation. The best binding peptides may be sequenced to identify key residues within one or more structurally related families of peptides. The peptide sequences may also suggest which residues may be safely replaced by alanine scanning or by mutagenesis at the DNA level. Mutagenesis libraries may be created and screened to further optimize the sequence of the best binders. See, e.g., WO 0024782, published May 4, 2000, and the references cited therein; U.S. Pat. No. 6,090,382 to Salfeld et al., issued Jul. 18, 2000; WO 93/06213, to Hoogenboom et al., published Apr. 1, 1993.

Other display library screening method are known as well. For example, E. coli displays employ a peptide library fused to either the carboxyl terminus of the lac-repressor or the peptidoglycan-associated lipoprotein, and expressed in E. coli. Ribosome display involves halting the translation of random RNAs prior to ribosome release, resulting in a library of polypeptides with their associated RNAs still attached. RNA-peptide screening employs chemical linkage of peptides to RNA. Additionally, chemically derived peptide libraries have been developed in which peptides are immobilized on stable, non-biological materials, such as polyethylene rods or solvent-permeable resins. Another chemically derived peptide library uses photolithography to scan peptides immobilized on glass slides. These methods of chemical-peptide screening may be advantageous because they allow use of D-amino acids and other unnatural analogues, as well as non-peptide elements. See WO 0024782, published May 4, 2000, and the references cited therein.

Moreover, structural analysis of protein-protein interaction may also be used to suggest peptides that mimic the binding activity of large protein ligands. In such an analysis, the crystal structure may suggest the identity and relative orientation of critical residues of the large protein ligand, from which a peptide may be designed. These analytical methods may also be used to investigate the interaction between a receptor protein and peptides selected by phage display, which may suggest further modification of the peptides to increase binding affinity. Thus, conceptually, one may discover peptide mimetics of any protein using phage display and the other methods mentioned above. For example, these methods provide for epitope mapping, for identification of critical amino acids in protein-protein interactions, and as leads for the discovery of new therapeutic agents. See WO 0024782, published May 4, 2000, and the references cited therein.

Additionally, target-binding moieties produced synthetically are another alternative or additional moiety that may be included in the pseudo-antibody constructs of the present invention. For example, solution-phase synthesis has been used to create the eptifibatide molecule that binds the platelet receptor glycoprotein IIb/IIIa of human platelets, thus inhibiting platelet aggregation. Eptifibatide, sold commercially as INTEGRILIN® (COR Therapeutics, Belmont, Calif.), is a cyclic heptapeptide containing six amino acids and one mercaptopropionyl (des-amino cycteinyl) residue. An interdisulfide bridge is formed between the cysteine amide and the mercaptopropionyl moieties. This synthetic peptide is bound to X as shown in Example 9, below, wherein X is or contains a functional group capable of forming the pseudo-antibody structure. The position of X is selected at any of those sites on the molecule at which substitution will retain some activity of the parent structure. In this specific example, the X may be a thiol group attached directly to the proline ring, or attached by way of an alkyl chain. X may also be carboxylic acid attached to the proline ring, or attached by way of an alkyl chain or any other functional group that would allow it to be attached covalently to the branching moiety that serves to construct the pseudo-antibody.

The nature and source of the target-binding moiety of the pseudo-antibody of the present invention is not limited. The following is a general discussion of the variety of proteins, peptides and biological molecules that may be used in the in accordance with the teachings herein. These descriptions do not serve to limit the scope of the invention, but rather illustrate the breadth of the invention.

Thus, an embodiment of the present invention may target one or more growth factors, or, conversely, derive the target-binding moiety from one or more growth factors. Briefly, growth factors are hormones or cytokine proteins that bind to receptors on the cell surface, with the primary result of activating cellular proliferation and/or differentiation. Many growth factors are quite versatile, stimulating cellular division in numerous different cell types; while others are specific to a particular cell-type. The following Table 1 presents several factors, but is not intended to be comprehensive or complete, yet introduces some of the more commonly known factors and their principal activities.

TABLE 1
Growth Factors

| Factor | Principal Source | Primary Activity | Comments |
|---|---|---|---|
| Platelet Derived | Platelets, endothelial | Promotes proliferation of | Dimer required for |
| Growth Factor | cells, placenta. | connective tissue, glial and | receptor binding. |
| (PDGF) | | smooth muscle cells. PDGF | Two different protein |
| | | receptor has intrinsic tyrosine | chains, A and B, form |
| | | kinase activity. | 3 distinct dimer |
| | | | forms. |
| Epidermal | Submaxillary gland, | promotes proliferation of | EGF receptor has |
| Growth Factor | Bnmnners gland. | mesenchymal, glial and | tyrosine kinase |
| (EGF) | | epithelial cells | activity, activated in |
| | | | response to EGF |
| | | | binding. |
| Fibroblast | Wide range of cells; | Promotes proliferation of | Four distinct |
| Growth Factor | protein is associated with | many cells including skeletal | receptors, all with |
| (FGF) | the ECM; nineteen family | and nervous system; inhibits | tyrosine kinase |
| | members. Receptors | some stem cells; induces | activity. FGF |
| | widely distributed in | mesodermal differentiation. | implicated in mouse |
| | bone, implicated in | Non-proliferative effects | mammary tumors and |
| | several bone-related | include regulation of pituitary | Kaposi's sarcoma. |
| | diseases. | and ovarian cell function. | |
| NGF | | Promotes neurite outgrowth | Several related |
| | | and neural cell survival | proteins first |
| | | | identified as proto- |
| | | | oncogenes; trkA |
| | | | (trackA), trkB, trkC |
| Erythropoietin | Kidney | Promotes proliferation and | Also considered a |
| (Epo) | | differentiation of erythrocytes | ‘blood protein,’ and a |
| | | | colony stimulating |
| | | | factor. |
| Transforming | Common in transformed | Potent keratinocyte growth | Related to EGF. |
| Growth Factor a | cells, found in | factor. | |
| (TGF-a) | macrophages and | | |
| | keratinocytes | | |
| Transforming | Tumor cells, activated | Anti-inflammatory (suppresses | Large family of |
| Growth Factor v | TH₁ cells (T-helper) and | cytokine production and class | proteins including |
| (TGF-b) | natural killer (NK) cells | II MHC expression), | activin, inhibin and |
| | | proliferative effects on many | bone morpho-genetic |
| | | mesenchymal and epithelial | protein. Several |
| | | cell types, may inhibit | classes and |
| | | macrophage and lymphocyte | subclasses of cell- |
| | | proliferation, | surface receptors |
| Insulin-Like | Primarily liver, produced | Promotes proliferation of | Related to IGF-II and |
| Growth Factor-I | in response to GH and | many cell types, autocrine and | proinsulin, also called |
| (IGF-I) | then induces subsequent | paracrine activities in addition | Somatomedin C. |
| | cellular activities, | to the initially observed | IGF-I receptor, like |
| | particularly on bone | endocrine activities on bone. | the insulin receptor, |
| | growth | | has intrinsic tyrosine |
| | | | kinase activity. IGF-I |
| | | | can bind to the |
| | | | insulin receptor. |
| Insulin-Like | Expressed almost | Promotes proliferation of | IGF-II receptor is |
| Growth | exclusively in embryonic | many cell types primarily of | identical to the |
| Factor-II | and neonatal tissues. | fetal origin. Related to IGF-I | mannose-6-phosphate |
| (IGF-II) | | and proinsulin. | receptor that is |
| | | | responsible for the |
| | | | integration of |
| | | | lysosomal enzymes |

Additional growth factors that may be produced in accordance with the present invention include Activin (Vale et al., 321 NATURE 776 (1986); Ling et al., 321 NATURE 779 (1986)), Inhibin (U.S. Pat. Nos. 4,737,578; 4,740,587), and Bone Morphongenic Proteins (BMPs) (U.S. Pat. No. 5,846,931; Wozney, CELLULAR & MOLECULAR BIOLOGY OF BONE 131-167 (1993).

In addition to the growth factors discussed above, the present invention may target or use other cytokines. Secreted primarily from leukocytes, cytokines stimulate both the humoral and cellular immune responses, as well as the activation of phagocytic cells. Cytokines that are secreted from lymphocytes are termed lymphokines, whereas those secreted by monocytes or macrophages are termed monokines. A large family of cytokines are produced by various cells of the body. Many of the lymphokines are also known as interleukins (ILs), because they are not only secreted by leukocytes, but are also able to affect the cellular responses of leukocytes. More specifically, interleukins are growth factors targeted to cells of hematopoietic origin. The list of identified interleukins grows continuously. See, e.g., U.S. Pat. No. 6,174,995; U.S. Pat. No. 6,143,289; Sallusto et al., 18 ANNU. REV. IMMUNol. 593 (2000) Kunkel et al., 59 J. LEUKOCYTE BIOL. 81 (1996).

Additional growth factor/cytokines encompassed in the present invention include pituitary hormones such as human growth hormone (HGH), follicle stimulating hormones (FSH, FSH α, and FSH β), Human Chorionic Gonadotrophins (HCG, HCG α, HCG β), uFSH (urofollitropin), Gonatropin releasing hormone (GRH), Growth Hormone (GH), leuteinizing hormones (LH, LH α, LH β), somatostatin, prolactin, thyrotropin (TSH, TSH α, TSH β), thyrotropin releasing hormone (TRH), parathyroid hormones, estrogens, progesterones, testosterones, or structural or functional analog thereof. All of these proteins and peptides are known in the art.

The cytokine family also includes tumor necrosis factors, colony stimulating factors, and interferons. See, e.g., Cosman, 7 BLOOD CELL (1996); Gruss et al., 85 BLOOD 3378 (1995); Beutler et al., 7 ANNU. REV. IMMUNOL. 625 (1989); Aggarwal et al., 260 J. BIOL. CHEM. 2345 (1985); Pennica et al., 312 NATURE 724 (1984); R & D Systems, CYTOKINE MINI-REVIEWS, at http://www.rndsystems.com.

Several cytokines are introduced, briefly, in Table 2 below.

TABLE 2
Cytokines

| Cytokine | Principal Source | Primary Activity |
|---|---|---|
| Interleukins | Primarily | Costimulation of APCs and T cells; |
| IL1-a and -b | macrophages but | stimulates IL-2 receptor |
| | also neutrophils, | production and expression |
| | endothelial cells, | of interferon-γ; may induce |
| | smooth muscle | proliferation in non-lymphoid cells. |
| | cells, glial | |
| | cells, astrocytes, | |
| | B- and T-cells, | |
| | fibroblasts, and | |
| | keratinocytes. | |
| IL-2 | CD4+ T-helper | Major interleukin responsible for |
| | cells, activated | clonal T-cell proliferation. IL-2 |
| | TH₁ cells, | also exerts effects on B-cells, |
| | NK cells. | macrophages, and natural killer |
| | | (NK) cells. IL-2 receptor is not |
| | | expressed on the surface of resting |
| | | T-cells, but expressed |
| | | constitutively on NK cells, that |
| | | will secrete TNF-a, IFN-g and |
| | | GM-CSF in response to IL-2, |
| | | which in turn activate |
| | | macrophages. |
| IL-3 | Primarily T-cells | Also known as multi-CSF, as it |
| | | stimulates stem cells to produce |
| | | all forms of hematopoietic cells. |
| IL-4 | TH₂ and mast | B cell proliferation, eosinophil |
| | cells | and mast cell growth and function, |
| | | IgE and class II MHC expression |
| | | on B cells, inhibition of monokine |
| | | production |
| IL-5 | TH₂ and mast | eosinophil growth and function |
| | cells | |
| IL-6 | Macrophages, | IL-6 acts in synergy with |
| | fibroblasts, | IL-1 and TNF-α in many immune |
| | endothelial cells | responses, including T-cell |
| | and activated | activation; primary inducer of the |
| | T-helper cells. | acute-phase response in liver; |
| | Does not induce | enhances the differentiation of |
| | cytokine | B-cells and their consequent |
| | expression. | production of immunoglobulin; |
| | | enhances Glucocorticoid synthesis. |
| IL-7 | thymic and | T and B lymphopoiesis |
| | marrow stromal | |
| | cells | |
| IL-8 | Monocytes, | Chemoattractant (chernokine) for |
| | neutrophils, | neutrophils, basophils and T-cells; |
| | macrophages, and | activates neutrophils to |
| | NK cells. | degranulate. |
| IL-9 | T cells | hematopoietic and thymopoietic |
| | | effects |
| IL-10 | activated TH₂ | inhibits cytokine production, |
| | cells, CD8⁺ T and | promotes B cell proliferation |
| | B cells, | and antibody production, |
| | macrophages | suppresses cellular immunity, |
| | | mast cell growth |
| IL-11 | stromal cells | synergisitc hematopoietic and |
| | | thrombopoietic effects |
| IL-12 | B cells, | proliferation of NK cells, INF-g |
| | macrophages | production, promotes cell-mediated |
| | | immune functions |
| IL-13 | TH₂ cells | IL-4-like activities |
| IL-18 | macrophages/ | Interferon-gamma-inducing factor |
| | Kupffer cells, | with potent pro-inflammatory |
| | keratinocytes, | activity |
| | glucocorticoid- | |
| | secreting adrenal | |
| | cortex cells, and | |
| | osteoblasts | |
| IL-21 | Activated T cells | IL21 has a role in proliferation |
| | | and maturation of natural killer |
| | | (NK) cell populations from bone |
| | | marrow, in the proliferation of |
| | | mature B-cell populations |
| | | co-stimulated with anti-CD40, |
| | | and in the proliferation of T cells |
| | | co-stimulated with anti-CD3. |
| IL-23 | Activated | A complex of p19 and the p40 |
| | dendritic cells | subunit of IL-12. IL-23 binds to |
| | | IL-12R beta 1 but not IL-12R |
| | | beta 2; activates Stat4 in PHA blast |
| | | T cells; induces strong proliferation |
| | | of mouse memory T cells; |
| | | stimulates IFN-gamma production |
| | | and proliferation in PHA blast |
| | | T cells, as well as in CD45RO |
| | | (memory) T cells. |
| TumorNecrosis | Primarily | Once called cachectin; induces |
| Factor | activated | the expression of other autocrine |
| TNF-α | macrophages. | growth factors, increases cellular |
| | | responsiveness to growth factors; |
| | | induces signaling pathways that |
| | | lead to proliferation; induces |
| | | expression of a number of nuclear |
| | | proto-oncogenes as well as of |
| | | several interleukins. |
| (TNF-β) | T-lymphocytes, | Also called lymphotoxin; |
| | particularly | kills a number of different cell |
| | cytotoxic | types, induces terminal |
| | T-lymphocytes | differentiation in others; inhibits |
| | (CTL cells); | lipoprotein lipase present on the |
| | induced by IL-2 | surface of vascular endothelial |
| | and antigen-T- | cells. |
| | Cell receptor | |
| | interactions. | |
| Interferons | macrophages, | Known as type I |
| INF-a and -b | neutrophils and | interferons; antiviral |
| | some somatic | effect; induction of |
| | cells | class I MHC on all |
| | | somatic cells; activation of |
| | | NK cells and macrophages. |
| Interferon | Primarily CD8+ | Type II interferon; induces of |
| INF-γ | T-cells, activated | class I MHC on all somatic cells |
| | TH₁ and NK cells | induces class II MHC on APCs and |
| | | somatic cells, activates |
| | | macrophages, neutrophils, NK |
| | | cells, promotes cell-mediated |
| | | immunity, enhances ability of |
| | | cells to present antigens to |
| | | T-cells; antiviral effects. |
| Monocyte | Peripheral blood | Attracts monocytes to sites of |
| Chemoattractant | monocytes/ | vascular endothelial cell |
| Protein-1 | macrophages | injury, implicated in |
| (MCP1) | | atherosclerosis. |
| Colony | | Stimulate the proliferation of |
| Stimulating | | specific pluripotent stem cells of |
| Factors (CSFs) | | the bone marrow in adults. |
| Granulocyte- | | Specific for proliferative effects on |
| CSF (G-CSF) | | cells of the granulocyte lineage; |
| | | proliferative effects on both classes |
| | | of lymphoid cells. |
| Macrophage- | | Specific for cells of the |
| CSF (M-CSF) | | macrophage lineage. |
| Granulocyte- | | Proliferative effects on cells of |
| MacrophageCSF | | both the macrophage and |
| (GM-CSF) | | granulocyte lineages. |

Other cytokines of interest that may be produced by the invention described herein include adhesion molecules(R & D Systems, ADHESION MOLECULES I (1996), available at http://www.rndsystems.com); angiogenin (U.S. Pat. No. 4,721,672; Moener et al., 226 EUR. J. BIOCHEM. 483 (1994)); annexin V (Cookson et al., 20 GENOMICS 463 (1994); Grundmann et al., 85 PROC. NATL. ACAD. Sci. USA 3708 (1988); U.S. Pat. No. 5,767,247); caspases (U.S. Pat. No. 6,214,858; Thornberry et al., 281 SCIENCE 1312 (1998)); chemokines (U.S. Pat. Nos. 6,174,995; 6,143,289; Sallusto et al., 18 ANNU. REV. IMMUNol. 593 (2000) Kunkel et al., 59 J. LEUKOCYTE BIOL. 81 (1996)); endothelin (U.S. Pat. Nos. 6,242,485; 5,294,569; 5,231,166); eotaxin (U.S. Pat. No. 6,271,347; Ponath et al., 97(3) J. CLIN. INVEST. 604-612 (1996)); Flt-3 (U.S. Pat. No. 6,190,655); heregulins (U.S. Pat. Nos. 6,284,535; 6,143,740; 6,136,558; 5,859,206; 5,840,525); Leptin (Leroy et al., 271(5) J. BIOL. CREM. 2365 (1996); Maffei et al., 92 PNAS 6957 (1995); Zhang Y. et al. (1994) NATURE 372: 425-432); Macrophage Stimulating Protein (MSP) (U.S. Pat. Nos. 6,248,560; 6,030,949; 5,315,000); Neurotrophic Factors (U.S. Pat. Nos. 6,005,081; 5,288,622); Pleiotrophin/Midkine (PTN/MK) (Pedraza et al., 117 J. BIOCHEM. 845 (1995); Tamura et al., 3 ENDOCRINE 21 (1995); U.S. Pat. No. 5,210,026; Kadomatsu et al., 151 BIOCHEM. BIOPHYS. RES. COMMUN. 1312 (1988)); STAT proteins (U.S. Pat. Nos. 6,030,808; 6,030,780; Darnell et al., 277 SCIENCE 1630-1635 (1997)); Tumor Necrosis Factor Family (Cosman, 7 BLOOD CELL (1996); Gruss et al., 85 BLOOD 3378 (1995); Beutler et al., 7 ANNU. REV. IMMUNOL. 625 (1989); Aggarwal et al., 260 J. BIOL. CHEM. 2345 (1985); Pennica et al., 312 NATURE 724 (1984).

Also of interest regarding cytokines are proteins or chemical moieties that interact with cytokines, such as Matrix Metalloproteinases (MMPs) (U.S. Pat. No. 6,307,089; NAGASE, MATRIX METALLOPROTEINASES IN ZINC METALLOPROTEASES IN HEALTH AND DISEASE (1996)), and Nitric Oxide Synthases (NOS) (Fukuto, 34 ADV. PHARM 1 (1995); U.S. Pat. No. 5,268,465).

The present invention may also be used to affect blood proteins, a generic name for a vast group of proteins generally circulating in blood plasma, and important for regulating coagulation and clot dissolution. See, e.g., Haematologic Technologies, Inc., HTI CATALOG, available at www.haemtech.com. Table 3 introduces, in a non-limiting fashion, some of the blood proteins contemplated by the present invention.

TABLE 3
Blood Proteins

| Protein | Principle Activity | Reference |
|---|---|---|
| Factor V | In coagulation, this | Mann et al., 57 ANN. REV. |
| | glycoprotein pro- | BIOCHEM. 915 (1988); see |
| | cofactor, is converted | also Nesheim et al., 254 J. BIOL. |
| | to active cofactor, | CHEM. 508 (1979); Tracy et al., |
| | factor Va, via the | 60 BLOOD 59 (1982); Nesheim |
| | serine protease α- | et al., 80 METHODS ENZYMOL. |
| | thrombin, and less | 249 (1981); Jenny et al., |
| | efficiently by its | 84 PROC. NATL. ACAD. SCI. |
| | serine protease | USA 4846 (1987). |
| | cofactor Xa. The | |
| | prothrombinase | |
| | complex rapidly | |
| | converts zymogen | |
| | prothrombin to the | |
| | active serine protease, | |
| | α-thrombin. Down | |
| | regulation of | |
| | prothrombinase | |
| | complex occurs via | |
| | inactivation of Va | |
| | by activated protein C. | |
| Factor VII | Single chain glyco- | See generally, Broze et al., |
| | protein zymogen in its | 80 METHODS ENZYMOL. 228 |
| | native form. | (1981); Bajaj et al., 256 J. BIOL. |
| | Proteolytic activation | CHEM. 253 (1981); Williams |
| | yields enzyme factor | et al., 264 J. BIOL. CHEM. 7536 |
| | VIIa, which binds to | (1989); Kisiel et al., |
| | integral membrane | 22 THROMBOSIS RES. 375 |
| | protein tissue factor, | (1981); Seligsohn et al., |
| | forming an enzyme | 64 J. CLIN. INVEST. 1056 |
| | complex that | (1979); Lawson et al., |
| | proteolytically | 268 J. BIOL. CHEM. 767 (1993). |
| | converts factor X to | |
| | Xa. Also known as | |
| | extrinsic factor | |
| | Xase complex. | |
| | Conversion of VII to | |
| | VIIa catalyzed by a | |
| | number of proteases | |
| | including thrombin, | |
| | factors IXa, Xa, | |
| | XIa, and XIIa. Rapid | |
| | activation also occurs | |
| | when VII combines | |
| | with tissue factor | |
| | in the presence of Ca, | |
| | likely initiated by a | |
| | small amount of pre- | |
| | existing VIIa. Not | |
| | readily inhibited by | |
| | antithrombin III/ | |
| | heparin alone, but is | |
| | inhibited when tissue | |
| | factor added. | |
| Factor IX | Zymogen factor IX, a | Thompson, 67 BLOOD, 565 |
| | single chain vitamin | (1986); Hedner et al., |
| | K-dependent | HEMOSTASIS AND |
| | glycoprotein, made in | THROMBOSIS 39-47 (R. W. |
| | liver. Binds to | Colman, J. Hirsh, V. J. Marder, |
| | negatively charged | E. W. Salzman ed., 2nd ed. |
| | phospholipid surfaces. | J. P. Lippincott Co., Philadelphia) |
| | Activated by factor | 1987; Fujikawa et al., |
| | XIα or the factor | 45 METHODS IN |
| | VIIa/tissue factor/ | ENZYMOLOGY 74 (1974). |
| | phospholipid complex. | |
| | Cleavage at one site | |
| | yields the intermediate | |
| | IXa, subsequently | |
| | converted to fully | |
| | active form IXaβ by | |
| | cleavage at another | |
| | site. Factor IXaβ is | |
| | the catalytic | |
| | component of the | |
| | “intrinsic | |
| | factor Xase complex” | |
| | (factor VIIIa/IXa/Ca²⁺/ | |
| | phospholipid) that | |
| | proteolytically | |
| | activates factor X to | |
| | factor Xa. | |
| Factor X | Vitamin K-dependent | See Davie et al., 48 ADV. |
| | protein zymogen, | ENZYMOL 277 (1979); Jackson, |
| | made in liver, | 49 ANN. REV. BIOCHEM. 765 |
| | circulates in plasma as | (1980); see also Fujikawa et al., |
| | a two chain molecule | 11 BIOCHEM. 4882 (1972); |
| | linked by a disulfide | Discipio et al., 16 BIOCHEM. |
| | bond. Factor Xa | 698 (1977); Discipio et al., |
| | (activated X) serves | 18 BIOCHEM. 899 (1979); |
| | as the enzyme | Jackson et al., 7 BIOCHEM. |
| | component of | 4506 (1968); McMullen et al., |
| | prothrombinase | 22 BIOCHEM. 2875 (1983). |
| | complex, responsible | |
| | for rapid conversion | |
| | of prothrombin to | |
| | thrombin. | |
| Factor XI | Liver-made glyco- | Thompson et al., 60 J. CLIN. |
| | protein homodimer | INVEST. 1376 (1977); Kurachi et |
| | circulates, in a | al., 16 BIOCHEM. 5831 (1977); |
| | non-covalent complex | Bouma et al., 252 J. BIOL. |
| | with high molecular | CHEM. 6432 (1977); Wuepper, |
| | weight kininogen, as a | 31 FED. PROC. 624 (1972); |
| | zymogen, requiring | Saito et al., 50 BLOOD 377 |
| | proteolytic activation | (1977); Fujikawa et al., 25 |
| | to acquire serine | BIOCHEM. 2417 (1986); Kurachi |
| | protease activity. | et al., 19 BIOCHEM. 1330 (1980); |
| | Conversion of factor | Scott et al., 69 J. CLIN. INVEST. |
| | XI to factor XIa is | 844 (1982). |
| | catalyzed by factor | |
| | XIIa. XIa unique | |
| | among the serine | |
| | proteases, since it | |
| | contains two active | |
| | sites per molecule. | |
| | Works in the intrinsic | |
| | coagulation pathway | |
| | by catalyzing | |
| | conversion of factor | |
| | IX to factor IXa. | |
| | Complex form, factor | |
| | XIa/HMWK, activates | |
| | factor XII to factor | |
| | XIIa and prekallikrein | |
| | to kallikrein. Major | |
| | inhibitor of XIa is a₁- | |
| | antitrypsin and to | |
| | lesser extent, anti- | |
| | thrombin-III. | |
| | Lack of factor | |
| | XI procoagulant | |
| | activity causes | |
| | bleeding disorder: | |
| | plasma thromboplastin | |
| | antecedent deficiency. | |
| Factor XII | Glycoprotein | Schmaier et al., 18-38, and Davie, |
| (Hageman | zymogen. Reciprocal | 242-267 HEMOSTASIS & |
| Factor) | activation of XII to | THROMBOSIS (Colman et al., |
| | active serine protease | eds., J. B. Lippincott Co., |
| | factor XIIa by | Philadelphia, 1987). |
| | kallikrein is central | |
| | to start of intrinsic | |
| | coagulation pathway. | |
| | Surface bound α-XIIa | |
| | activates factor XI | |
| | to XIa. Secondary | |
| | cleavage of α-XIIa by | |
| | kallikrein yields | |
| | β-XIIa, and catalyzes | |
| | solution phase | |
| | activation of | |
| | kallikrein, factor | |
| | VII and the classical | |
| | complement cascade. | |
| Factor XIII | Zymogenic form of | See McDonaugh, 340-357 |
| | glutaminyl-peptide γ- | HEMOSTASIS & THROMBOSIS |
| | glutamyl transferase | (Colman et al., eds., J. B. |
| | factor XIIIa | Lippincott Co., Philadelphia, |
| | (fibrinoligase, plasma | 1987); Folk et al., 113 METHODS |
| | transglutaminase, | ENZYMOL. 364 (1985); |
| | fibrin stabilizing | Greenberg et al., 69 BLOOD 867 |
| | factor). Made in the | (1987). Other proteins known to be |
| | liver, found | substrates for Factor XIIIa, that |
| | extracellularly in | may be hemostatically important, |
| | plasma and intra- | include fibronectin (Iwanaga et al., |
| | cellularly in platelets, | 312 ANN. NY ACAD. SCI. 56 |
| | megakaryocytes, | (1978)), a₂- antiplasmin |
| | monocytes, placenta, | (Sakata et al., 65 J. CLIN. |
| | uterus, liver and | INVEST. 290 (1980)), collagen |
| | prostrate tissues. | (Mosher et al., 64 J. CLIN. |
| | Circulates as a | INVEST. 781 (1979)), factor V |
| | tetramer of 2 pairs of | (Francis et al., 261 J. BIOL. |
| | nonidentical subunits | CHEM. 9787 (1986)), von |
| | (A₂B₂). Full | Willebrand Factor (Mosher et al., |
| | expression of activity | 64 J. CLIN. INVEST. 781 (1979)) |
| | is achieved only after | and thrombospondin (Bale et al., |
| | the Ca²⁺- and | 260 J. BIOL. CHEM. 7502 (1985); |
| | fibrin(ogen)- | Bohn, 20 MOL. CELL |
| | dependent dissociation | BIOCHEM. 67 (1978)). |
| | of B subunit dimer | |
| | from A₂’ dimer. | |
| | Last of the zymogens | |
| | to become activated in | |
| | the coagulation | |
| | cascade, the only | |
| | enzyme in this | |
| | system that is not | |
| | a serine protease. | |
| | XIIIa stabilizes the | |
| | fibrin clot by cross- | |
| | linking the α and | |
| | γ-chains of fibrin. | |
| | Serves in cell | |
| | proliferation in | |
| | wound healing, | |
| | tissue remodeling, | |
| | atherosclerosis, and | |
| | tumor growth. | |
| Fibrinogen | Plasma | FURLAN, Fibrinogen, IN |
| | fibrinogen, a large | HUMAN PROTEIN DATA, |
| | glycoprotein, disulfide | (Haeberli, ed., VCH Publishers, |
| | linked dimer made of | N.Y., 1995); Doolittle, in |
| | 3 pairs of non- | HAEMOSTASIS & THROM- |
| | identical chains (Aa, | BOSIS, 491-513 (3rd ed., Bloom |
| | Bb and g), made | et al., eds., Churchill Livingstone, |
| | in liver. | 1994); HANTGAN, et al., in |
| | Aa has N-terminal | HAEMOSTASIS & THROM- |
| | peptide (fibrinopeptide | BOSIS 269-89 (2d ed., Forbes |
| | A (EPA), factor XIIIa | et al., eds., Churchill Livingstone, |
| | crosslinking sites, and | 1991). |
| | 2 phosphorylation | |
| | sites. Bb has | |
| | fibrinopeptide B | |
| | (FPB), 1 of 3 N-linked | |
| | carbohydrate moieties, | |
| | and an N-terminal | |
| | pyroglutamic acid. | |
| | The g chain contains | |
| | the other N-linked | |
| | glycos. site, and | |
| | factor XIIla cross- | |
| | linking sites. Two | |
| | elongated subunits | |
| | ((AaBbg)₂) align in | |
| | an antiparallel way | |
| | forming a trinodular | |
| | arrangement of the | |
| | 6 chains. Nodes | |
| | formed by disulfide | |
| | rings between the | |
| | 3 parallel chains. | |
| | Central node | |
| | (n-disulfide knot, E | |
| | domain) formed by | |
| | N-termini of all 6 | |
| | chains held together | |
| | by 11 disulfide | |
| | bonds, contains the | |
| | 2 IIa-sensitive sites. | |
| | Release of FPA by | |
| | cleavage generates | |
| | Fbn I, exposing a | |
| | polymerization site on | |
| | Aa chain. These sites | |
| | bind to regions on | |
| | the D domain of Fbn | |
| | to form proto- | |
| | fibrils. Subsequent | |
| | IIa cleavage of FPB | |
| | from the Bb chain | |
| | exposes additional | |
| | polymerization sites, | |
| | promoting lateral | |
| | growth of Fbn | |
| | network. Each of the 2 | |
| | domains between the | |
| | central node and | |
| | the C-terminal nodes | |
| | (domains D and E) | |
| | has parallel a-helical | |
| | regions of the Aa, | |
| | Bb and g chains | |
| | having protease- | |
| | (plasmin-) sensitive | |
| | sites. Another major | |
| | plasmin sensitive site | |
| | is in hydrophilic | |
| | preturbance of a-chain | |
| | from C-terminal | |
| | node. Controlled | |
| | plasmin degradation | |
| | converts Fbg into | |
| | fragments D and E. | |
| Fibronectin | High molecular | Skorstengaard et al., 161 Fur. J. |
| | weight, adhesive, | BIOCHEM. 441 (1986); |
| | glycoprotein found in | Kornblihtt et al., 4 EMBO J. 1755 |
| | plasma and extra- | (1985); Odermatt et al., 82 PNAS |
| | cellular matrix in | 6571 (1985); Hynes, R.O., ANN. |
| | slightly different | REV. CELL BIOL., 1, 67 (1985); |
| | forms. Two peptide | Mosher 35 ANN. REV. MED. 561 |
| | chains interconnected | (1984); Rouslahti et al., 44 Cell |
| | by 2 disulfide bonds, | 517 (1986); Hynes 48 CELL 549 |
| | has 3 different | (1987); Mosher 250 BIOL. CHEM. |
| | types of repeating | 6614 (1975). |
| | homologous sequence | |
| | units. Mediates cell | |
| | attachment by | |
| | interacting with cell | |
| | surface receptors and | |
| | extracellular matrix | |
| | components. Contains | |
| | an Arg-Gly-Asp-Ser | |
| | (RGDS) cell | |
| | attachment- | |
| | promoting sequence, | |
| | recognized by | |
| | specific cell | |
| | receptors, such as | |
| | those on platelets. | |
| | Fibrin-fibronectin | |
| | complexes stabilized | |
| | by factor XIIIa- | |
| | catalyzed covalent | |
| | cross-linking of | |
| | fibronectin to | |
| | the fibrin a chain. | |
| b₂ | Also called b₂I and | See, e.g., Lozier et al., 81 PNAS |
| Glycoprotein | Apolipoprotein H. | 2640-44 (1984); Kato & Enjyoi 30 |
| I | Highly glycosylated | BIOCHEM. 11687-94 (1997); |
| | single chain protein | Wurm, 16 INT'L J. BIOCHEM. |
| | made in liver. Five | 511-15 (1984); Bendixen et al., |
| | repeating mutually | 31 BIOCHEM. 3611-17 (1992); |
| | homologous domains | Steinkasserer et al., 277 |
| | consisting of | BIOCHEM. J. 387-91 (1991); |
| | approximately 60 | Nimpf et al., 884 BIOCHEM. |
| | amino acids disulfide | BIOPHYS. ACTA 142-49 (1986); |
| | bonded to form Short | Kroll et. al. 434 BIOCHEM. |
| | Consensus Repeats | BIOPHYS. Acta 490-501 (1986); |
| | (SCR) or Sushi | Polz et al., 11 INT'L J. |
| | domains. Associated | BIOCHEM. 265-73 (1976); |
| | with lipoproteins, | McNeil et al., 87 PNAS |
| | binds anionic | 4120-24 (1990); Galli et a;. |
| | surfaces like anionic | II LANCET 1544-47 (1990); |
| | vesicles, platelets, | Matsuuna et al., I LANCET |
| | DNA, mitochondria, | 177-78 (1990); Pengo et al., |
| | and heparin. Binding | 73 THROMBOSIS & |
| | can inhibit contact | HAEMOSTASIS 29-34 (1995). |
| | activation pathway in | |
| | blood coagulation. | |
| | Binding to activated | |
| | platelets inhibits | |
| | platelet associated | |
| | prothrombinase and | |
| | adenylate cyclase | |
| | activities. Complexes | |
| | between b21 and | |
| | cardiolipin have | |
| | been implicated in | |
| | the anti-phospholipid | |
| | related immune | |
| | disorders LAC and | |
| | SLE. | |
| Osteonectin | Acidic, | Villarreal et al., 28 BIOCHEM. |
| | noncollagenous | 6483 (1989); Tracy et al., |
| | glycoprotein | 29 INT'L J. BIOCHEM. 653 |
| | (Mr = 29,000) | (1988); Romberg et al., 25 |
| | originally isolated | BIOCHEM. 1176 (1986); Sage & |
| | from fetal and adult | Bornstein 266 J. BIOL. CHEM. |
| | bovine bone matrix. | 14831 (1991); Kelm & Mann 4 J. |
| | May regulate bone | BONE MIN. RES. 5245 (1989); |
| | metabolism by binding | Kelm et al., 80 BLOOD 3112 |
| | hydroxyapatite to | (1992). |
| | collagen. Identical | |
| | to human placental | |
| | SPARC. An alpha | |
| | granule component of | |
| | human platelets | |
| | secreted during | |
| | activation. A | |
| | small portion of | |
| | secreted osteonectin | |
| | expressed on the | |
| | platelet cell surface in | |
| | an activation- | |
| | dependent manner | |
| Plasminogen | Single chain | See Robbins, 45 METHODS IN |
| | glycoprotein zymogen | ENZYMOLOGY 257 (1976); |
| | with 24 disulfide | COLLEN, 243-258 BLOOD |
| | bridges, no free | COAG. (Zwaal et al., eds., |
| | sulfhydryls, and 5 | New York, Elsevier, 1986); see |
| | regions of internal | also Castellino et al., |
| | sequence homology, | 80 METHODS IN |
| | “kringles”, each five | ENZYMOLOGY 365 (1981); |
| | triple-looped, three | Wohl et al., 27 THROMB. RES. |
| | disulfide bridged, | 523 (1982); Barlow et al., 23 |
| | and homologous to | BIOCHEM. 2384 (1984); |
| | kringle domains in | SOTTRUP-JENSEN ET AL., |
| | t-PA, u-PA and | 3 PROGRESS IN CHEM. |
| | prothrombin. Inter- | FIBRINOLYSIS & |
| | action of plasminogen | THROMBOLYSIS 197-228 |
| | with fibrin and α2- | (Davidson et al., eds., Raven |
| | antiplasmin is | Press, New York 1975). |
| | mediated by lysine | |
| | binding sites. | |
| | Conversion of | |
| | plasminogen to | |
| | plasmin occurs by | |
| | variety of mechanisms, | |
| | including urinary type | |
| | and tissue type | |
| | plasminogen | |
| | activators, | |
| | streptokinase, | |
| | staphylokinase, | |
| | kallikrein, factors IXa | |
| | and XIIa, but all | |
| | result in hydrolysis at | |
| | Arg560-Val561, | |
| | yielding two chains | |
| | that remain covalently | |
| | associated by a | |
| | disulfide bond. | |
| tissue | t-PA, a serine | See Plasminogen. |
| Plasminogen | endopeptidase | |
| Activator | synthesized by | |
| | endothelial cells, is | |
| | the major physiologic | |
| | activator of | |
| | plasminogen in | |
| | clots, catalyzing | |
| | conversion of | |
| | plasminogen to | |
| | plasmin by hydrolising | |
| | a specific arginine- | |
| | alanine bond. | |
| | Requires fibrin | |
| | for this activity, | |
| | unlike the kidney- | |
| | produced version, | |
| | urokinase-PA. | |
| Plasmin | See Plasminogen. | See Plasininogen. |
| | Plasmin, a serine | |
| | protease, cleaves | |
| | fibrin, and activates | |
| | and/or degrades | |
| | compounds of | |
| | coagulation, kinin | |
| | generation, and | |
| | complement systems. | |
| | Inhibited by a | |
| | number of plasma | |
| | protease inhibitors in | |
| | vitro. Regulation of | |
| | plasmin in vivo | |
| | occurs mainly through | |
| | interaction with | |
| | a₂-antiplasmin, and to | |
| | a lesser extent, a₂- | |
| | macroglobulin. | |
| Platelet | Low molecular weight, | Rucinski et al., 53 BLOOD 47 |
| Factor-4 | heparin-binding | (1979); Kaplan et al., 53 BLOOD |
| | protein secreted | 604 (1979); George 76 BLOOD |
| | from agonist-activated | 859 (1990); Busch et al., 19 |
| | platelets as a | THROMB. RES. 129 (1980); Rao |
| | homotetramer in | et al., 61 BLOOD 1208 (1983); |
| | complex with a high | Brindley, et al., 72 J. CLIN. |
| | molecular weight, | INVEST. 1218 (1983); Deuel et |
| | proteoglycan, carrier | al., 74 PNAS 2256 (1981); |
| | protein. Lysine-rich, | Osterman et al., 107 BIOCHEM. |
| | COOH-terminal region | BIOPHYS. RES. COMMUN. 130 |
| | interacts with cell | (1982); Capitanio et al., 839 |
| | surface expressed | BIOCHEM. BIOPHYS. ACTA |
| | heparin-like | 161 (1985). |
| | glycosaminoglycans | |
| | on endothelial cells, | |
| | PF-4 neutralizes | |
| | anticoagulant activity | |
| | of heparin exerts | |
| | procoagulant effect, | |
| | and stimulates release | |
| | of histamine from | |
| | basophils. Chemotactic | |
| | activity toward | |
| | neutrophils and | |
| | monocytes. Binding | |
| | sites on the platelet | |
| | surface have been | |
| | identified and may be | |
| | important for platelet | |
| | aggregation. | |
| Protein C | Vitamin K-dependent | See Esmon, 10 PROGRESS IN |
| | zymogen, protein C, | THROMB. & HEMOSTS. 25 |
| | made in liver as a | (1984); Stenflo, 10 SEMIN. IN |
| | single chain poly- | THROMB. & HEMOSTAS. 109 |
| | peptide then converted | (1984); Griffen et al., |
| | to a disulfide | 60 BLOOD 261 (1982); Kisiel et |
| | linked heterodimer. | al., 80 METHODS ENZYMOL. |
| | Cleaving the heavy- | 320 (1981); Discipio et al., 18 |
| | chain of human | BIOCHEM. 899 (1979). |
| | protein C converts | |
| | the zymogen into the | |
| | serine protease, | |
| | activated protein C. | |
| | Cleavage catalyzed | |
| | by a complex of α- | |
| | thrombin and | |
| | thrombomodulin. | |
| | Unlike other vitamin | |
| | K dependent coagula- | |
| | tion factors, | |
| | activated protein C is | |
| | an anticoagulant | |
| | that catalyzes the | |
| | proteolytic inactivation | |
| | of factors Va and | |
| | VIIIa, and contributes | |
| | to the fibrinolytic | |
| | response by complex | |
| | formation with | |
| | plasminogen | |
| | activator inhibitors. | |
| Protein S | Single chain vitamin | Walker 10 SEMIN. THROMB. |
| | K-dependent protein | HEMOSTAS. 131 (1984); |
| | functions in | Dahlback et al., 10 SEMIN. |
| | coagulation and | THROMB. HEMOSTAS., 139 |
| | complement cascades. | (1984); Walker 261 J. BIOL. |
| | Does not possess the | CHEM. 10941 (1986). |
| | catalytic triad. | |
| | Complexes to C4b | |
| | binding protein | |
| | (C4BP) and to | |
| | negatively charged | |
| | phospholipids, | |
| | concentrating C4BP at | |
| | cell surfaces | |
| | following injury. | |
| | Unbound S serves as | |
| | anticoagulant cofactor | |
| | protein with | |
| | activated Protein C. | |
| | A single cleavage | |
| | by thrombin abolishes | |
| | protein S cofactor | |
| | activity by removing | |
| | gla domain. | |
| Protein Z | Vitamin K-dependent, | Sejima et al., 171 BIOCHEM. |
| | single-chain protein | BIOPHYSICS RES. COMM. |
| | made in the liver. | 661 (1990); Hogg et al., 266 J. |
| | Direct requirement for | BIOL. CHEM. 10953 (1991); |
| | the binding of | Hogg et al., 17 BIOCHEM. |
| | thrombin to | BIOPHYSICS RES. COMM. 801 |
| | endothelial | (1991); Han et al., 38 BIOCHEM. |
| | phospholipids. Domain | 11073 (1999); Kemkes-Matthes |
| | structure similar | et al., 79 THROMB. RES. 49 |
| | to that of other | (1995). |
| | vitamin K-dependant | |
| | zymogens like | |
| | factors VII, IX, X, | |
| | and protein C. N- | |
| | tenninal region | |
| | contains carboxy- | |
| | glutamic acid domain | |
| | enabling phospholipid | |
| | membrane binding. | |
| | C-terminal region | |
| | lacks “typical” serine | |
| | protease activation | |
| | site. | |
| | Cofactor for | |
| | inhibition of | |
| | coagulation factor Xa | |
| | by serpin called | |
| | protein Z-dependant | |
| | protease inhibitor. | |
| | Patients diagnosed | |
| | with protein Z | |
| | deficiency have | |
| | abnormal bleeding | |
| | diathesis during | |
| | and after surgical | |
| | events. | |
| Prothrombin | Vitamin K-dependent, | Mann et al., 45 METHODS IN |
| | single-chain protein | ENZYMOLOGY 156 (1976); |
| | made in the liver. | Magnusson et al., PROTEASES IN |
| | Binds to negatively | BIOLOGICAL CONTROL |
| | charged phospholipid | 123-149 (Reich et al., eds. |
| | membranes. Contains | Cold Spring Harbor Labs., New |
| | two “kringle” | York 1975); Discipio et al., |
| | structures. Mature | 18 BIOCHEM. 899 (1979). |
| | protein circulates | |
| | in plasma as a | |
| | zymogen and, | |
| | during coagulation, is | |
| | proteolytically | |
| | activated to the | |
| | potent serine | |
| | protease α-thrombin. | |
| α-Thrombin | See Prothrombin. | METHODS ENZYMOL. 156 |
| | During coagulation, | (1976). |
| | thrombin cleaves | |
| | fibrinogen to form | |
| | fibrin, the terminal | |
| | proteolytic step in | |
| | coagulation, forming | |
| | the fibrin clot. | |
| | Thrombin also | |
| | responsible for | |
| | feedback activation of | |
| | procofactors V and | |
| | VIII. | |
| | Activates factor XIII | |
| | and platelets, | |
| | functions as | |
| | vasoconstrictor | |
| | protein. | |
| | Procoagulant activity | |
| | arrested by | |
| | heparin cofactor II or | |
| | the antithrombin | |
| | IlI/heparin complex, | |
| | or complex | |
| | formation with | |
| | thrombomodulin. | |
| | Formation of | |
| | thrombin/ | |
| | thrombomodulin | |
| | complex results in | |
| | inability of thrombin | |
| | to cleave fibrinogen | |
| | and activate factors | |
| | V and VIII, but | |
| | increases the | |
| | efficiency | |
| | of thrombin for | |
| | activation of the | |
| | anticoagulant, protein | |
| | C. | |
| b-Thrombo- | Low molecular weight, | See, e.g., George 76 BLOOD 859 |
| globulin | heparin-binding, | (1990); Holt & Niewiarowski |
| | platelet-derived | 632 BIOCHIM. BIOPHYS. ACTA |
| | tetramer protein, | 284 (1980); Niewiarowski et al., |
| | consisting of four | 55 BLOOD 453 (1980); Varma |
| | identical peptide- | et al., 701 BIOCHIM. BIOPHYS. |
| | chains. Lower affinity | ACTA 7 (1982); Senior et al., |
| | for heparin than PF-4. | 96 J. CELL. BIOL. 382 (1983). |
| | Chemotactic activity | |
| | for human fibroblasts, | |
| | other functions | |
| | unknown. | |
| Thrombo- | Human TPO | Horikawa et al., 90(10) BLOOD |
| poietin | (Thrombopoietin, Mpl- | 4031-38 (1997); de Sauvage et al., |
| | ligand, MGDF) | 369 NATURE 533-58 (1995). |
| | stimulates the | |
| | proliferation and | |
| | maturation of | |
| | megakaryocytes and | |
| | promotes increased | |
| | circulating levels of | |
| | platelets in vivo. | |
| | Binds to c-Mpl | |
| | receptor. | |
| Thrombo- | High-molecular | Dawes et al., 29 THROMB. |
| spondin | weight, heparin- | RES. 569 (1983); Switalska et. |
| | binding glycoprotein | al., 106 J. LAB. CLIN. MED. 690 |
| | constituent of | (1985); Lawler et al. 260 J. |
| | platelets, consisting | BIOL. CHEM. 3762 (1985); |
| | of three, identical, | Wolff et al., 261 J. BIOL. |
| | disulfide-linked | CHEM. 6840 (1986); Asch et al., |
| | polypeptide chains. | 79 J. CLIN. CHEM. 1054 (1987); |
| | Binds to surface of | Jaffe et al., 295 NATURE |
| | resting and activated | 246 (1982); Wright et al., 33 J. |
| | platelets, may effect | HISTOCHEM. CYTOCHEM. 295 |
| | platelet adherence and | (1985); Dixit et al., 259 J. |
| | aggregation. An | BIOL. CHEM. 10100 (1984); |
| | integral component of | Mumby et al., 98 J. CELL. |
| | basement membrane in | BIOL. 646 (1984); Lahav et al, |
| | different tissues. | 145 EUR. J. BIOCHEM. 151 |
| | Interacts with a | (1984); Silverstein et al, 260 J. |
| | variety of extra- | BIOL. CHEM. 10346 (1985); |
| | cellular macro- | Clezardin et al. 175 EUR. J. |
| | molecules including | BIOCHEM. 275 (1988); Sage & |
| | heparin, collagen, | Bornstein (1991). |
| | fibrinogen and | |
| | fibronectin, | |
| | plasminogen, | |
| | plasminogen | |
| | activator, and | |
| | osteonectin. May | |
| | modulate cell-matrix | |
| | interactions. | |
| Von | Multimeric plasma | Hoyer 58 BLOOD 1 (1981); |
| Willebrand | glycoprotein made of | Ruggeri & Zimmerman 65 J. |
| Factor | identical subunits held | CLIN. INVEST. 1318 (1980); |
| | together by disulfide | Hoyer & Shainoff 55 BLOOD |
| | bonds. During normal | 1056 (1980); Meyer et al., 95 J. |
| | hemostasis, larger | LAB. CLIN. INVEST. 590 (1980); |
| | multimers of vWF | Santoro 21 THROMB. RES. |
| | cause platelet plug | 689 (1981); Santoro, & Cowan 2 |
| | formation by forming | COLLAGEN RELAT. RES. 31 |
| | a bridge between | (1982); Morton et al., 32 |
| | platelet glycoprotein | THROMB. RES. 545 (1983); |
| | IB and exposed | Tuddenham et al., 52 BRIT. |
| | collagen in the | J. HAEMATOL. 259 (1982). |
| | subendothelium. | |
| | Also binds and | |
| | transports factor | |
| | VIII (antihemophilic | |
| | factor) in plasma. | |

Additional blood proteins contemplated herein include the following human serum proteins, which may also be placed in another category of protein (such as hormone or antigen): Actin, Actinin, Amyloid Serum P, Apolipoprotein E, B2-Microglobulin, C-Reactive Protein (CRP), Cholesterylester transfer protein (CETP), Complement C3B, Ceruplasmin, Creatine Kinase, Cystatin, Cytokeratin 8, Cytokeratin 14, Cytokeratin 18, Cytokeratin 19, Cytokeratin 20, Desmin, Desmocollin 3, FAS (CD95), Fatty Acid Binding Protein, Ferritin, Filamin, Glial Filament Acidic Protein, Glycogen Phosphorylase Isoenzyme BB (GPBB), Haptoglobulin, Human Myoglobin, Myelin Basic Protein, Neurofilament, Placental Lactogen, Human SHBG, Human Thyroid Peroxidase, Receptor Associated Protein, Human Cardiac Troponin C, Human Cardiac Troponin I, Human Cardiac Troponin T, Human Skeletal Troponin I, Human Skeletal Troponin T, Vimentin, Vinculin, Transferrin Receptor, Prealbumin, Albumin, Alpha-1-Acid Glycoprotein, Alpha-1-Antichymotrypsin, Alpha-1-Antitrypsin, Alpha-Fetoprotein, Alpha-1-Microglobulin, Beta-2-microglobulin, C-Reactive Protein, Haptoglobulin, Myoglobulin, Prealbumin, PSA, Prostatic Acid Phosphatase, Retinol Binding Protein, Thyroglobulin, Thyroid Microsomal Antigen, Thyroxine Binding Globulin, Transferrin, Troponin I, Troponin T, Prostatic Acid Phosphatase, Retinol Binding Globulin (RBP). All of these proteins, and sources thereof, are known in the art. Many of these proteins are available commercially from, for example, Research Diagnostics, Inc. (Flanders, N.J.).

The pseudo-antibody of the present invention may also incorporate or target neurotransmitters, or functional portions thereof. Neurotransmitters are chemicals made by neurons and used by them to transmit signals to the other neurons or non-neuronal cells (e.g., skeletal muscle; myocardium, pineal glandular cells) that they innervate. Neurotransmitters produce their effects by being released into synapses when their neuron of origin fires (i.e., becomes depolarized) and then attaching to receptors in the membrane of the post-synaptic cells. This causes changes in the fluxes of particular ions across that membrane, making cells more likely to become depolarized, if the neurotransmitter happens to be excitatory, or less likely if it is inhibitory. Neurotransmitters can also produce their effects by modulating the production of other signal-transducing molecules (“second messengers”) in the post-synaptic cells. See generally COOPER, BLOOM & ROTH, THE BIOCHEMICAL BASIS OF NEUROPHARMACOLOGY (7th Ed. Oxford Univ. Press, NYC, 1996); http://web.indstate.edu/thcme/mwking/nerves. Neurotransmitters contemplated in the present invention include, but are not limited to, Acetylcholine, Serotonin, γ-aminobutyrate (GABA), Glutamate, Aspartate, Glycine, Histamine, Epinephrine, Norepinephrine, Dopamine, Adenosine, ATP, Nitric oxide, and any of the peptide neurotransmitters such as those derived from pre-opiomelanocortin (POMC), as well as antagonists and agonists of any of the foregoing.

Numerous other proteins or peptides may serve as either targets, or as a source of target-binding moieties as described herein. Table 4 presents a non-limiting list and description of some pharmacologically active peptides which may serve as, or serve as a source of a functional derivative of, a portion of a pseudo-antibody of the present invention.

TABLE 4
Pharmacologically active peptides

| Binding | | |
|---|---|---|
| partner/ | | |
| Protein of | Pharmaco- | |
| interest (form | logical | |
| of peptide) | activity | Reference |
| EPO receptor | EPO mimetic | Wrighton et al., 273 SCIENCE 458-63 |
| (intrapeptide | | (1996); U.S. Pat. No. 5,773,569, issued |
| disulfide- | | Jun. 30, 1998. |
| bonded) | | |
| EPO receptor | EPO mimetic | Livnah et al., 273 SCIENCE 464-71 |
| (C-terminally | | (1996); Wrighton et al., 15 NATURE |
| cross-linked | | BIOTECHNOLOGY 1261-5 (1997); |
| dimer) | | Int'l Patent Application WO 96/40772, |
| | | published Dec. 19, 1996. |
| EPO receptor | EPO mimetic | Naranda et al., 96 PNAS 7569-74 |
| (linear) | | (1999). |
| c-Mpl | TPO-mimetic | Cwirla et al., 276 SCIENCE 1696-9 |
| (linear) | | (1997); U.S. Pat. No. 5,869,451, issued |
| | | Feb. 9, 1999; U.S. Pat. No. 5,932,946, |
| | | issued Aug. 3, 1999. |
| c-Mpl | TPO-mimetic | Cwirla et al., 276 SCIENCE 1696-9 |
| (C-terminally | | (1997). |
| cross-linked | | |
| dimer) | | |
| (disulfide- | stimulation of | Paukovits et al., 364 HOPPE- |
| linked dimer) | hematopoesis | SEYLERS Z. PHYSIOL. CHEM. |
| | (“G-CSF- | 30311 (1984); Laerurngal., 16 EXP. |
| | mimetic”) | HEMAT. 274-80 (1988). |
| (alkylene- | G-CSF- | Batnagar et al., 39 J. MED. CHEM. |
| linked dimer) | mimetic | 38149 (1996); Cuthbertson et al., |
| | | 40 J. MED. CHEM. 2876-82 (1997); |
| | | King et al., 19 EXP. HEMATOL. 481 |
| | | (1991); King et al., 86(Suppl. 1) |
| | | BLOOD 309 (1995). |
| IL-1 receptor | inflammatory | U.S. Pat. No. 5,608,035; U.S. Pat. No. |
| (linear) | and auto- | 5,786,331; U.S Pat. No. 5,880,096; |
| | immune | Yanofsky et al., 93 PNAS 7381-6 |
| | diseases | (1996); Akeson et al., 271 J. BIOL. |
| | (“IL-1 | CHEM. 30517-23 (1996); Wiekorek |
| | antagonist” | et al., 49 POL. J. PHARMACOL. |
| | or “IL-1 ra- | 107-17 (1997); Yanofsky, 93 PNAS |
| | mimetic”) | 7381-7386 (1996). |
| Facteur | stimulation of | Inagaki-Ohara et al., 171 |
| thyrnique | lymphocytes | CELLULAR IMMUNOL. 30-40 |
| (linear) | (FTS-mimetic) | (1996); Yoshida, 6 J. |
| | | IMMUNOPHARMACOL. 141-6 |
| | | (1984). |
| CTLA4 MAb | CTLA4- | Fukumoto et al., 16 NATURE |
| (intrapeptide | mimetic | BIOTECH. 267-70 (1998). |
| di-sulfide | | |
| bonded) | | |
| TNF-a receptor | TNF-a | Takasaki et al., 15 NATURE |
| (exo-cyclic) | antagonist | BIOTECH. 1266-70 (1997); WO |
| | | 98/53842, published Dec. 3, 1998. |
| TNF-a receptor | TNF-a | Chirinos-Rojas, J. IMM., 5621-26. |
| (linear) | antagonist | |
| C3b | inhibition of | Sahu et al., 157 IMMUNOL. |
| (intrapeptide | complement | 884-91 (1996); Morikis et al., 7 |
| di-sulfide | activation; | PROTEIN SCI. 619-27 (1998). |
| bonded) | autoinimune | |
| | diseases (C3b | |
| | antagonist) | |
| vinculin | cell adhesion | Adey et al., 324 BIOCHEM. J. 523-8 |
| (linear) | processes, cell | (1997). |
| | growth, | |
| | differentiation | |
| | wound healing, | |
| | tumor | |
| | metastasis | |
| | (“vinculin | |
| | binding”) | |
| C4 binding | anti-thrombotic | Linse et al. 272 BIOL. |
| protein | | CHEM. 14658-65 (1997). |
| (C413P) | | |
| (linear) | | |
| urokinase | processes | Goodson et al., 91 PNAS 7129-33 |
| receptor | associated with | (1994); International patent application |
| (linear) | urokinase inter- | WO 97/35969, published Oct. 2, 1997. |
| | action with its | |
| | receptor (e.g. | |
| | angiogenesis, | |
| | tumor cell | |
| | invasion and | |
| | metastasis; | |
| | (URK | |
| | antagonist) | |
| Mdm2, Hdm2 | Inhibition of | Picksley et al., 9 ONCOGENE 2523-9 |
| (linear) | inactivation | (1994); Bottger et al. 269 J. MOL. |
| | of p53 | BIOL. 744-56 (1997); Bottger et al., 13 |
| | mediated by | ONCOGENE 13: 2141-7 (1996). |
| | Mdm2 or | |
| | hdm2; | |
| | anti-tumor | |
| | (“Mdm/ | |
| | hdm | |
| | antagonist”) | |
| p21^{WAF1} | anti-tumor by | Ball et al., 7 CURR. BIOL. |
| (linear) | mimicking the | 71-80 (1997). |
| | activity of | |
| | p21^{WAF1} | |
| farnesyl | anti-cancer by | Gibbs et al., 77 CELL 175-178 (1994). |
| transferase | preventing | |
| (linear) | activation of | |
| | ras oncogene | |
| Ras effector | anti-cancer | Moodie et at., 10 TRENDS GENEL |
| domain | by inhibiting | 44-48 (1994); Rodriguez et al., |
| (linear) | biological | 370 NATURE 527-532 (1994). |
| | function of | |
| | the ras | |
| | oncogene | |
| SH2/SH3 | anti-cancer by | Pawson et al, 3 CURR. BIOL. 434-432 |
| domains | inhibiting | (1993); Yu et al., 76 CELL 933-945 |
| (linear) | tumor growth | (1994). |
| | with activated | |
| | tyrosine | |
| | kinases | |
| p16^{INK4} | anti-cancer by | Fahraeus et al., 6 CURR. BIOL. 84-91 |
| (linear) | mimicking | (1996). |
| | activity of | |
| | p16; e.g., | |
| | inhibiting | |
| | cyclin D-Cdk | |
| | complex | |
| | (“p, 16- | |
| | mimetic”) | |
| Src, Lyn | inhibition of | Stauffer et al., 36 BIOCHEM. 9388-94 |
| (linear) | Mast cell | (1997). |
| | activation, | |
| | IgE-related | |
| | conditions, | |
| | type I | |
| | hypersensitivity | |
| | (“Mast cell | |
| | antagonist”). | |
| Mast cell | treatment of | International patent application WO |
| protease | inflammatory | 98/33812, published Aug. 6, 1998. |
| (linear) | disorders | |
| | mediated by | |
| | release of | |
| | tryptase-6 | |
| | (“Mast | |
| | cell protease | |
| | inhibitors”) | |
| SH3 domains | treatment of | Rickles et al., 13 EMBO J. 5598- |
| (linear) | SH3-mediated | 5604 (1994); Sparks et al., 269 J. |
| | disease states | BIOL. CHEM. 238536 (1994); |
| | (“SH3 | Sparks et al., 93 PNAS 1540-44 |
| | antagonist”) | (1996). |
| HBV core | treatment of | Dyson & Muray, PNAS 2194-98 |
| antigen | HBV viral | (1995). |
| (HBcAg) | antigen | |
| (linear) | (HBcAg) | |
| | infections | |
| | (“anti-HBV”) | |
| selectins | neutrophil | Martens et al., 270 J. BIOL. |
| (linear) | adhesion | CHEM. 21129-36 (1995); |
| | inflammatory | European Pat. App. EP 0714 |
| | diseases | 912, published Jun. 5, 1996. |
| | (“selectin | |
| | antagonist”) | |
| calmodulin | calmodulin | Pierce et al., 1 MOLEC. |
| (linear, | antagonist | DIVEMILY 25965 (1995); |
| cyclized) | | Dedman et al., 267 J. BIOL. |
| | | CHEM. 23025-30 (1993); Adey & |
| | | Kay, 169 GENE 133-34 |
| | | (1996). |
| integrins | tumor-homing; | International patent applications WO |
| (linear, | treatment for | 95/14714, published Jun. 1, 1995; WO |
| cyclized) | conditions | 97/08203, published Mar. 6, 1997; WO |
| | related to | 98/10795, published Mar. 19, 1998; |
| | integrin- | WO 99/24462, published May 20, 1999; |
| | mediated | Kraft et al., 274 J. BIOL. CHEM. |
| | cellular events, | 1979-85 (1999). |
| | including | |
| | platelet | |
| | aggregation, | |
| | thrombosis, | |
| | wound healing, | |
| | osteoporosis, | |
| | tissue repair, | |
| | angiogenesis | |
| | (e.g., for | |
| | treatment of | |
| | cancer) | |
| | and tumor | |
| | invasion | |
| | (“integrin- | |
| | binding”) | |
| fibronectin and | treatment of | International patent application WO |
| extracellular | inflammatory | 98/09985, published Mar. 12, 1998. |
| matrix | and | |
| components of | autoimmune | |
| T-cells and | conditions | |
| macrophages | | |
| (cyclic, linear) | | |
| somatostatin | treatment or | European patent application EP 0 911 |
| and cortistatin | prevention of | 393, published Apr. 28, 1999. |
| (linear) | hormone- | |
| | producing | |
| | tumors, | |
| | acromegaly, | |
| | giantism, | |
| | dementia, | |
| | gastric ulcer, | |
| | tumor growth, | |
| | inhibition of | |
| | hormone | |
| | secretion, | |
| | modulation of | |
| | sleep or | |
| | neural activity | |
| bacterial | antibiotic; | U.S. Pat. No. 5,877,151, issued Mar. 2, |
| lipopoly- | septic shock; | 1999. |
| saccharide | disorders | |
| (linear) | modulatable | |
| | by CAP37 | |
| parclaxin, | antipathogenic | International patent application WO |
| mellitin | | 97/31019, published Aug. 28, 1997. |
| (linear or | | |
| cyclic) | | |
| VIP | impotence, | International patent application WO |
| (linear, cyclic) | neuro- | 97/40070, published Oct. 30, 1997. |
| | degenerative | |
| | disorders | |
| CTLs | cancer | European patent application EP 0 770 |
| (linear) | | 624, published May 2, 1997. |
| THF-gamma2 | | Burnstein, 27 BIOCHEM. 4066-71 |
| (linear) | | (1988). |
| Amylin | | Cooper, 84 PNAS 8628-32 (1987). |
| (linear) | | |
| Adreno- | | Kitamura, 192 BBRC 553-60 (1993). |
| medullin | | |
| (linear) | | |
| VEGF | anti- | Fairbrother. 37 BIOCHEM. 17754-64 |
| (cyclic, | angiogenic; | (1998). |
| linear) | cancer, | |
| | rheumatoid | |
| | arthritis, | |
| | diabetic | |
| | retinopathy, | |
| | psoriasis | |
| | (“VEGF | |
| | antagonist”’) | |
| MMP | inflammation | Koivunen, 17 NATURE BIOTECH. |
| (cyclic) | and | 768-74 (1999). |
| | autoimmune | |
| | disorders; | |
| | tumor growth | |
| | (“MMP | |
| | inhibitor”) | |
| HGH | | U.S. Pat. No. 5,869,452, issued |
| fragment | | Feb. 9, 1999. |
| (linear) | | |
| Echistatin | inhibition of | Gan, 263 J. BIOL. 19827-32 (1988). |
| | platelet | |
| | aggregation | |
| SLE | SLE | International patent application WO |
| autoantibody | | 96/30057, published Oct. 3, 1996. |
| (linear) | | |
| GDI alpha | suppression of | Ishikawa et al., 1 FEBS LETT. 20-4 |
| | tumor | (1998). |
| | metastasis | |
| anti- | endothelial cell | Blank Mal., 96 PNAS 5164-8 (1999). |
| phospholipid | activation, | |
| β-2 | anti- | |
| glycoprotein-1 | phospholipid | |
| (β2GPI) | syndrome | |
| antibodies | (APS), | |
| | thrombo- | |
| | embolic | |
| | phenomena, | |
| | thrombocyto- | |
| | penia, and | |
| | recurrent | |
| | fetal loss | |
| T-Cell | diabetes | International patent application WO |
| Receptor | | 96/101214, published Apr. 18, 1996. |
| β chain | | |
| (linear) | | |
| EPO receptor | EPO mimetic | Wrighton et al. (1996), Science 273: |
| (intrapeptide | | 458-63; U.S. Pat. No. 5,773,569, issued |
| disulfide- | | Jun. 30, 1998 to Wrighton et al. |
| bonded) | | |
| EPO receptor | EPO mimetic | Livnah et al. (1996), Science 273: 464- |
| (C-terminally | | 71; Wrighton et al. (1997), Nature |
| cross-linked | | Biotechnology 15:1261-5; int'l patent |
| dimer) | | application WO 96/40772, published |
| | | Dec. 19, 1996 |
| EPO receptor | EPO mimetic | Naranda et al., 96 PNAS 7569-74 |
| (linear) | | (1999) |
| c-Mpl | TPO-mimetic | Cwirla et al.(1997) Science 276:1696-9; |
| (linear) | | U.S. Pat. No. 5,869,451, issued Feb. |
| | | 9, 1999; U.S. Pat. No. 5,932,946, |
| | | issued Aug. 3, 1999 |
| c-Mpl | TPO-mimetic | Cwirla et al. (1997) Science |
| (C-terminally | | 276:1696-9 |
| cross-linked | | |
| dimer) | | |
| (disulfide- | stimulation of | Paukovits et al. |
| linked | hematopoesis | (1984), Hoppe-Seylers Z. Physiol. |
| dimer) | (“G-CSF- | Chem. 365: 30311; Laerurn gal. |
| | mimetic”) | (1988), Exp. Hemat. 16:274-80 |
| (alkylene- | G-CSF- | Batnagar 91-al. (1996), linked dimer J. |
| linked dimer) | mimetic | Med. Chem. 39:38149; Cuthbertson |
| | | et al. (1997), J. Med. Chem. |
| | | 40:2876-82; King et al. (1991), |
| | | Exp. Hematol. 19:481; King |
| | | et al. (1995), Blood 86 (Suppl. 1): 309 |
| IL-1 receptor | inflammatory | U.S. Pat. No. 5,608,035; U.S. Pat. No. |
| (linear) | and | 5,786,331; U.S. Pat. No. 5,880,096; |
| | autoimmune | Yanofsky 91-al. (1996) PNAS |
| | diseases (“IL-1 | 93:7381-6; Akeson et al. |
| | antagonist” or | (1996), J. Biol. Chem. 271: |
| | “IL-1 ra- | 30517-23; Wiekzorek et al. (1997), |
| | mimetic”) | Pol. J. Pharmacol. 49:107-17; Yanofsky |
| | | (1996), PNAs, 93:7381-7386. |
| Facteur | stimulation of | Inagaki-Ohara et al. (1996), Cellular |
| thyrnique | lymphocytes | Immunol. 171: 30-40; Yoshida (1984), |
| (linear) | (FTS-mimetic) | J. Immunopharmacol, 6:141-6. |
| CTLA4 MAb | CTLA4- | Fukumoto et al. (1998), Nature Biotech. |
| (intrapeptide | mimetic | 16:267-70 |
| di-sulfide | | |
| bonded) | | |
| TNF-a receptor | TNF-a | Takasaki et al. (1997), Nature Biotech. |
| (exo-cyclic) | antagonist | 15:1266-70; WO 98/53842, published |
| | | Dec. 3, 1998. |
| TNF-a receptor | TNF-a | Chirinos-Rojas J. Imm., 5621-26. |
| (linear) | antagonist | |
| C3b | inhibition of | Sahu et al. (1996), Immunol. |
| (intrapeptide | complement | 157:884-91; Morikis et al. (1998), |
| di-sulfide | activation; | Protein Sci. 7:619-27. |
| bonded) | autoimmune | |
| | diseases (C3b | |
| | antagonist) | |
| vinculin | cell adhesion | Adey et al. (1997), Biochem. J. |
| (linear) | processes, cell | 324:523-8 |
| | growth, | |
| | differentiation | |
| | wound healing, | |
| | tumor | |
| | metastasis | |
| | (“vinculin | |
| | binding”) | |
| C4 binding | anti- | Linse et al. 272 Biol. Chem. |
| protein | thrombotic | 14658-65 (1997) |
| (C413P) | | |
| (linear) | | |
| urokinase | processes | Goodson et al. (1994), 91 PNAS |
| receptor | associated with | 7129-33; International |
| (linear) | urokinase | application WO 97/35969, |
| | interaction with | published Oct. 2, 1997 |
| | its receptor | |
| | (e.g. | |
| | angiogenesis, | |
| | tumor cell | |
| | invasion and | |
| | metastasis; | |
| | (URK | |
| | antagonist) | |
| Mdm2, Hdm2 | Inhibition of | Picksley et al. (1994), Oncogene 9: |
| (linear) | inactivation of | 2523-9; Bottger et al. (1997) J. Mol. |
| | p53 mediated | Biol. 269:744-56; Bottger et al. (1996), |
| | by Mdm2 or | Oncogene 13:2141-7 |
| | hdm2; anti- | |
| | tumor | |
| | (“Mdm/hdm | |
| | antagonist”) | |
| p21^{WAF1} | anti-tumor by | Ball et al. (1997), Cuff. Biol. 7:71-80. |
| (linear) | mimicking the | |
| | activity of | |
| | p21^{WAF1} | |
| farnesyl | anti-cancer by | Gibbs et al. (1994). Cell 77:175-178 |
| transferase | preventing | |
| (linear) | activation of | |
| | ras oncogene | |
| Ras effector | anti-cancer | Moodie et at. (1994), Trends Genel |
| domain | by inhibiting | 10:44-48 Rodriguez et al. (1994), |
| (linear) | biological | Nature 370:527-532. |
| | function of | |
| | the ras | |
| | oncogene | |
| SH2/SH3 | anti-cancer by | Pawson et al (1993), Cuff. Biol. 3:434- |
| domains | inhibiting | 432, Yu et al. (1994), Cell 76:933-945. |
| (linear) | tumor growth | |
| | with activated | |
| | tyrosine | |
| | kinases | |
| p16^{INK4} | anti-cancer by | Fahraeus et al. (1996), Curr. Biol. |
| (linear) | mimicking | 6:84-91 |
| | activity of | |
| | p16; e.g., | |
| | inhibiting | |
| | cyclin D-Cdk | |
| | complex (“p, | |
| | 16-mimetic”) | |
| Src, Lyn | inhibition of | Stauffer et al. (1997), Biochem. |
| (linear) | Mast cell | 36:9388-94. |
| | activation, | |
| | IgE-related | |
| | conditions, | |
| | type I | |
| | hypersensitivity | |
| | (“Mast cell | |
| | antagonist”). | |
| Mast cell | treatment of | International application WO 98/33812, |
| protease | inflammatory | published Aug. 6, 1998 |
| (linear) | disorders | |
| | mediated by | |
| | release of | |
| | tryptase-6 | |
| | (“Mast cell | |
| | protease | |
| | inhibitors”) | |
| SH3 domains | treatment of | Rickles et al. (1994), EMBO J. |
| (linear) | SH3-mediated | 13:5598-5604; Sparks aLal. |
| | disease states | (1994), J. Biol. Chem. 269: |
| | (“SH3 | 238536; Sparks et al. (1996), |
| | antagonist”) | PNAS 93:1540-44. |
| HBV core | treatment of | Dyson & Muray (1995), Proc. |
| antigen | HBV viral | Natl. Acad. Sci. 92:2194-98. |
| (HBcAg) | antigen | |
| (linear) | (HBcAg) | |
| | infections | |
| | (“anti-HBV”) | |
| selectins | neutrophil | Martens et al. (1995), J. Biol. |
| (linear) | adhesion | Chem. 270: 21129-36; European |
| | inflammatory | pat. app. EP 0 714 912, published |
| | diseases | Jun. 5, 1996 |
| | (“selectin | |
| | antagonist”) | |
| calmodulin | calmodulin | Pierce et al. (1995), Molec. |
| (linear, | antagonist | Divemily 1:25965; Dedman et |
| cyclized) | | al. (1993), J. Biol. Chem. 268: |
| | | 23025-30; Adey & Kay (1996), |
| | | Gene 169:133-34. |
| integrins | tumor-homing; | International applications WO |
| (linear, | treatment for | 95/14714, published Jun. 1, 1995; |
| cyclized) | conditions | WO 97/08203, published |
| | related to | Mar. 6, 1997; WO 98/10795, |
| | integrin- | published Mar. 19, 1998; |
| | mediated | WO 99/24462, published May 20, 1999; |
| | cellular events, | Kraft et al. (1999), J. Biol. Chem. |
| | including | 274:1979-85. |
| | platelet | |
| | aggregation, | |
| | thrombosis, | |
| | wound healing, | |
| | osteoporosis, | |
| | tissue repair, | |
| | angiogenesis | |
| | (e.g., for | |
| | treatment of | |
| | cancer) | |
| | and tumor | |
| | invasion | |
| | (“integrin- | |
| | binding”) | |
| fibronectin | treatment of | WO 98/09985. published |
| and extra- | inflammatory | Mar. 12, 1998. |
| cellular matrix | and | |
| components of | autoimmune | |
| T-cells | conditions | |
| and macro- | | |
| phages | | |
| (cyclic, | | |
| linear) | | |
| somatostatin | treatment or | European patent application 0 911 393, |
| and | prevention of | published Apr. 28, 1999. |
| cortistatin | hormone- | |
| (linear) | producing | |
| | tumors, | |
| | acromegaly, | |
| | giantism, | |
| | dementia, | |
| | gastric ulcer, | |
| | tumor growth, | |
| | inhibition of | |
| | hormone | |
| | secretion, | |
| | modulation of | |
| | sleep or | |
| | neural activity | |
| bacterial | antibiotic; | U.S. Pat. No. 5,877,151, issued |
| lipopoly- | septic shock; | Mar. 2, 1999. |
| saccharide | disorders | |
| (linear) | modulatable | |
| | by CAP37 | |
| parciaxin, | antipathogenic | WO 97/31019, published Aug. 28, |
| mellitin | | 1997. |
| (linear or | | |
| cyclic) | | |
| VIP | impotence, | WO 97/40070, published Oct. 30, |
| (linear, | neuro- | 1997. |
| cyclic) | degenerative | |
| | disorders | |
| CTLs | cancer | EP 0 770 624, published May 2, 1997. |
| (linear) | | |
| THF-gamma2 | | Burnstein (1988), Biochem., |
| (linear) | | 27:4066-71 |
| Amylin | | Cooper (1987), PNAS 84:8628-32. |
| (linear) | | |
| Adreno- | | Kitamura (1993), BBRC, 192:553-60 |
| medullin | | |
| (linear) | | |
| VEGF | anti- | Fairbrother (1998), Biochem., |
| (cyclic, | angiogenic; | 37:17754-64. |
| linear) | cancer, | |
| | rheumatoid | |
| | arthritis, | |
| | diabetic | |
| | retinopathy, | |
| | psoriasis | |
| | (“VEGF | |
| | antagonist”’) | |
| MMP | inflammation | Koivunen 17 Nature Biotech., 768-74 |
| (cyclic) | and auto- | (1999). |
| | immune | |
| | disorders; | |
| | tumor growth | |
| | (“MMP | |
| | inhibitor”) | |
| HGH | | U.S. Pat. No. 5,869,452. |
| fragment | | |
| (linear) | | |
| Echistatin | inhibition of | Gan (1988), J. Biol. 263:19827-32. |
| | platelet | |
| | aggregation | |
| SLE | SLE | WO 96/30057, published Oct. 3, 1996. |
| autoantibody | | |
| (linear) | | |
| GDI alpha | suppression of | Ishikawa et al., 1 FEBS Lett. 20-4 |
| | tumor | (1998). |
| | metastasis | |
| anti- | endothelial | Blank Mal. (1999), PNAS 96:5164-8. |
| phospholipid | cell activation, | |
| β-2 | anti- | |
| glycoprotein-1 | phospholipid | |
| (β2GP1) | syndrome | |
| antibodies | (APS), | |
| | throm- | |
| | boembolic | |
| | phenomena, | |
| | thrombocyto- | |
| | penia, and | |
| | recurrent | |
| | fetal loss | |
| T-Cell | diabetes | WO 96/101214, published |
| Receptor | | Apr. 18, 1996. |
| β chain (linear) | | |

There are two pivotal cytokines in the pathogenesis of rheumatoid arthritis, IL-1 and TNF-α. They act synergistically to induce each other, other cytokines, and COX-2. Research suggests that IL-1 is a primary mediator of bone and cartilage destruction in rheumatoid arthritis patients, whereas TNF-α appears to be the primary mediator of inflammation.

In a preferred embodiment of the invention, the pseudo-antibody comprises a target-binding moiety that binds to tumor necrosis factor alpha (TNFα), a pro-inflamatory cytokine. U.S. Pat. No. 6,277,969, issued Aug. 21, 2001; U.S. Pat. No. 6,090,382, issued Jul. 10, 2000. Anti-TNFα antibodies have shown great promise as therapeutics. For example, Infliximab, provided commercially as REMICADE® by Centocor, Inc. (Malvern, Pa.) has been used for the treatment of several chronic autoimmune diseases such as Crohn's disease and rheumatoid arthritis. Treacy, 19(4) HUM. EXP. TOXICOL. 226-28 (2000); see also Chantry, 2(1) CURR. OPIN. ANTI-INFLAMMATORY IMMUNOMODULATORY INVEST. DRUGS 31-34 (2000); Rankin et al., 34(4) BRIT. J. RHEUMATOLOGY 334-42 (1995). Preferably, any exposed amino acids of the TNFα-binding moiety of the pseudo-antibody are those with minimal antigenicity in humans, such as human or humanized amino acid sequences. These moieties may be generated by screening libraries, as described above, by grafting human amino acid sequences onto murine-derived paratopes (Siegel et al., 7(1) CYTOKINE 15-25 (1995); WO 92/11383, published Jul. 9, 1992) or monkey-derived paratopes (WO 93/02108, published Feb. 4, 1993), or by utilizing xenomice (WO 96/34096, published Oct. 31, 1996). Alternatively, murine-derived anti-TNFα antibodies have exhibited efficacy. Saravolatz et al., 169(1) J. INFECT. DIS. 214-17 (1994).

Alternatively, instead of being derived from an antibody, the TNFα binding moiety of the pseudo-antibody may be derived from the TNFα receptor. For example, Etanercept is a recombinant, soluble TNFα receptor molecule that is administered subcutaneously and binds to TNFα in the patient's serum, rendering it biologically inactive. Etanercept is a dimeric fusion protein consisting of the extracellular ligand-binding portion of the human 75 kilodalton (p75) tumor necrosis factor receptor (TNFR) linked to the Fc portion of human IgG1. The Fc component of etanercept contains the C_{H}2 domain, the C_{H}3 domain and hinge region, but not the C_{H}1 domain of IgG1. Etanercept is produced by recombinant DNA technology in a Chinese hamster ovary (CHO) mammalian cell expression system. It consists of 934 amino acids and has an apparent molecular weight of approximately 150 kilodaltons. Etanercept may be obtained as ENBREL™, manufactured by Immunex Corp. (Seattle, Wash.). Etanercept may be efficacious in rheumatoid arthritis. Hughes et al., 15(6) BIODRUGS 379-93 (2001).

Another form of human TNF receptor exists as well, identified as p55. Kalinkovich et al., J. INFERON & CYTOKINE RES. 15749-57 (1995). This receptor has also been explored for use in therapy. See, e.g., Qian et al. 118 ARCH. OPHTHALMOL. 1666-71 (2000). A previous formulation of the soluble p55 TNF receptor had been coupled to polyethylene glycol [r-metHuTNFbp PEGylated dimer (TNFbp)], and demonstrated clinical efficacy but was not suitable for a chronic indication due to the development antibodies upon multiple dosing, which resulted in increased clearance of the drug. A second generation molecule was designed to remove the antigenic epitopes of TNFbp, and may be useful in treating patients with rheumatoid arthritis. Davis et al., Presented at the Ann. European Cong. Rheumatology, Nice, France (Jun. 21-24, 2000).

IL-1 receptor antagonist (IL-1Ra) is a naturally occurring cytokine antagonist that demonstrates anti-inflammatory properties by balancing the destructive effects of IL-1α and IL-1β in rheumatoid arthritis but does not induce any intracellular response. Hence, in a preferred embodiment of the invention, the pseudo-antibody comprises IL-1Ra, or any structural or functional analog thereof. Two structural variants of IL-1Ra exist: a 17-kDa form that is secreted from monocytes, macrophages, neutrophils, and other cells (sIL-1Ra) and an 18-kDa form that remains in the cytoplasm of keratinocytes and other epithelial cells, monocytes, and fibroblasts (icIL-1Ra). An additional 16-kDa intracellular isoform of IL-1Ra exists in neutrophils, monocytes, and hepatic cells. Both of the major isoforms of IL-1Ra are transcribed from the same gene through the use of alternative first exons. The production of IL-1Ra is stimulated by many substances including adherent IgG, other cytokines, and bacterial or viral components. The tissue distribution of IL-1Ra in mice indicates that sIL-1Ra is found predominantly in peripheral blood cells, lungs, spleen, and liver, while icIL-1Ra is found in large amounts in skin. Studies in transgenic and knockout mice indicate that IL-1Ra is important in host defense against endotoxin-induced injury. IL-1Ra is produced by hepatic cells with the characteristics of an acute phase protein. Endogenous IL-1Ra is produced in human autoimmune and chronic inflammatory diseases. The use of neutralizing anti-IL-1Ra antibodies has demonstrated that endogenous IL-1Ra is an important natural antiinflammatory protein in arthritis, colitis, and granulomatous pulmonary disease. Patients with rheumatoid arthritis treated with IL-1Ra for six months exhibited improvements in clinical parameters and in radiographic evidence of joint damage. Arend et al., 16 ANN. REV. IMMUNOL. 27-55 (1998).

Yet another example of an IL-1Ra that may be incorporated into the pseudo-antibody of the present invention is a recombinant human version called interleukin-1 17.3 Kd met-IL1ra, or Anakinra, produced by Amgen, (San Francisco, Calif.) under the name KINERET™. Anakinra has also shown promise in clinical studies involving patients with rheumatoid arthritis. Presented at the 65th Ann. Sci. Meeting of Am. College Rheumatology (Nov. 12, 2001).

Another embodiment of the pseudo-antibody includes a moiety that targets cyclooxigenase-2 (COX-2). COX-2 selective inhibitors-such as valdecoxib, etoricoxib, celecoxib and rofecoxib are less toxic to the gastrointestinal (GI) tract than conventional nonsteroidal anti-inflammatory drugs (NSAIDs), while possessing equivalent analgesic efficacy for conditions such as osteoarthritis (OA), rheumatoid arthritis (RA), dental pain and menstrual pain. In a preferred embodiment of the invention, a COX-2 inhibitor may be included in the pseudo-antibody construct with a TNFα antagonist. See, e.g., U.S. Pat. Nos. 5,474,995, 5,409,944.

In another embodiment of the invention, the pseudo-antibody includes a selective p38 Mitogen-Activated Protein Kinase (p38 MAP kinase) inhibitor. For example, the compound SB 242235 is a potent and selective p38 MAP kinase inhibitor. The compound is active in the adjuvant arthritic rat, where it inhibits inflammation and has significant joint-protective effects as measured by changes in bone mineral density, magnetic resonance imaging, micro-computed tomography, and histology. These studies indicate that cytokine-suppressing, low molecular weight p38 inhibitors may be orally active, disease-modifying agents in the treatment of rheumatoid arthritis. Badger et al, Disease-Modifying Activity of SB 242235, A Selective Inhibitor of p38 Mitogen-Activated Protein Kinase, in Rat Adjuvant-Induced Arthritis, Proceedings of the 1999 AACR, NCI, EORTC Int'l Conference, Am. Assoc. for Cancer Res.

In another embodiment of the invention, the pseudo-antibody comprises a target-binding moiety that binds interleukin 12 (IL-12), a heterodimeric cytokine consisting of glycosylated polypeptide chains of 35 and 40 kD which are disulfide bonded. The cytokine is synthesized and secreted by antigen presenting cells, including dendritic cells, monocytes, macrophages, B cells, Langerhans cells and keratinocytes, as well as natural killer (NK) cells. IL-12 mediates a variety of biological processes and has been referred to as NK cell stimulatory factor (NKSF), T-cell stimulating factor, cytotoxic T-lymphocyte maturation factor and EBV-transformed B-cell line factor. Curfs et al., 10 CLIN. MICRO. REV. 742-80 (1997). Interleukin-12 can bind to the IL-12 receptor expressed on the plasma membrane of cells (e.g., T cells, NK cell), thereby altering (e.g., initiating, preventing) biological processes. For example, the binding of IL-12 to the IL-12 receptor can stimulate the proliferation of pre-activated T cells and NK cells, enhance the cytolytic activity of cytotoxic T cells (CTL), NK cells and LAK (lymphokine activated killer) cells, induce production of gamma interferon (IFN GAMMA) by T cells and NK cells and induce differentiation of naive Th0 cells into Th1 cells that produce IFN GAMMA and IL-2. Trinchieri, 13 ANN. REV. IMMUNOLOGY 251-76 (1995). In particular, IL-12 is vital for the generation of cytolytic cells (e.g., NK, CTL) and for mounting a cellular immune response (e.g., a Th1 cell mediated immune response). Thus, IL-12 is critically important in the generation and regulation of both protective immunity (e.g., eradication of infections) and pathological immune responses (e.g., autoimmunity). Hendrzak et al., 72 LAB. INVESTIGATION 619-37 (1995). Accordingly, an immune response (e.g., protective or pathogenic) can be enhanced, suppressed or prevented by manipulation of the biological activity of IL-12 in vivo, for example, by means of an antibody.

In another embodiment of the present invention, the pseudo-antibody comprises or targets an integrin. Integrins have been implicated in the angiogenic process, by which tumor cells form new blood vessels that provide tumors with nutrients and oxygen, carry away waste products, and to act as conduits for the metastasis of tumor cells to distant sites, Gastl et al., 54 ONCOL. 177-84 (1997). Integrins are heterodimeric transmembrane proteins that play critical roles in cell adhesion to the extracellular matrix (ECM) which, in turn, mediates cell survival, proliferation and migration through intracellular signaling. During angiogenesis, a number of integrins that are expressed on the surface of activated endothelial cells regulate critical adhesive interactions with a variety of ECM proteins to regulate distinct biological events such as cell migration, proliferation and differentiation. Specifically, the closely related but distinct integrins a Vb3 and a Vb5 have been shown to mediate independent pathways in the angiogenic process. An antibody generated against αVβ3 blocked basic fibroblast growth factor (bFGF) induced angiogenesis, whereas an antibody specific to αVβ5 inhibited vascular endothelial growth factor-induced (VEGF-induced) angiogenesis. Eliceiri et al., 103 J. CLIN. INVEST. 1227-30 (1999); Friedlander et al., 270 SCIENCE 1500-02 (1995).

In another preferred embodiment of the invention, the pseudo-antibody comprises at least one glycoprotein IIb/IIIa receptor antagonist. More specifically, the final obligatory step in platelet aggregation is the binding of fibrinogen to an activated membrane-bound glycoprotein complex, GP IIb/IIIa. Platelet activators such as thrombin, collagen, epinephrine or ADP, are generated as an outgrowth of tissue damage. During activation, GP IIb/IIIa undergoes changes in conformation that results in exposure of occult binding sites for fibrinogen. There are six putative recognition sites within fibrinogen for GP IIb/IIIa and thus fibrinogen can potentially act as a hexavalent ligand to crossing GP IIb/IIIa molecules on adjacent platelets. A deficiency in either fibrinogen or GP IIb/IIIa a prevents normal platelet aggregation regardless of the agonist used to activate the platelets. Since the binding of fibrinogen to its platelet receptor is an obligatory component of normal aggregation, GP IIb/IIIa is an attractive target for an antithrombotic agent.

Results from clinical trials of GP IIb/IIIa inhibitors support this hypothesis. A Fab fragment of the monoclonal antibody 7E3, which blocks the GP IIb/IIIa receptor, has been shown to be an effective therapy for the high risk angioplasty population. It is used as an adjunct to percutaneous transluminal coronary angioplasty or atherectomy for the prevention of acute cardiac ischemic complications in patients at high risk for abrupt closure of the treated coronary vessel. Although 7E3 blocks both the IIb/IIIa receptor and the αᵥβ3 receptor, its ability to inhibit platelet aggregation has been attributed to its function as a IIb/IIIa receptor binding inhibitor. The IIb/IIIa receptor antagonist may be, but is not limited to, an antibody, a fragment of an antibody, a peptide, or an organic molecule. For example, the target-binding moiety may be derived from 7E3, an antibody with glycoprotein IIb/IIIa receptor antagonist activity. 7E3 is the parent antibody of c7E3, a Fab fragment known as abciximab, known commercially as REOPRO® produced by Centocor, Inc. (Malvern, Pa.). Abciximab binds and inhibits the adhesive receptors GPIIb/IIIa and αᵥβ3, leading to inhibition of platelet aggregation and thrombin generation, and the subsequent prevention of thrombus formation. U.S. Pat. Nos. 5,976,532, 5,877,006, 5,770,198; Coller, 78 THROM HAEMOST. 730-35 (1997); JORDAN ET AL., in ADHESION RECEPTORS AS THERAPEUTIC TARGETS 281-305 (Horton, ed. CRC Press, New York, 1996); Jordan et al., in NEW THERAPEUTIC AGENTS IN THROMBOSIS & THROMBOLYSIS (Sasahara & Loscalzo, eds. Marcel Kekker, Inc. New York, 1997).

Additionally, the glycoprotein IIb/IIIa receptor antagonist of the present invention may further comprise a thrombolytic. For example, the thrombolytic may be tPA, or a functional variation thereof. RETAVASE®, produced by Centocor, Inc. (Malvern, Pa.), is a variant tPA with a prolonged half-life. In mice, the combination of Retavase and the IIb/IIIa receptor antagonist c7E3 Fab markedly augmented the dissolution of pulmonary embolism. See Provisional Patent Application Serial No. 60/304409.

Alternative target-binding moieties envisioned in the present invention also include non-peptide molecules. For example, tirofiban hydrochloride is a non-peptide antagonist of the platelet glycoprotein IIb/IIIa receptor, that inhibits platelet aggregation. See U.S. Pat. No. 6,117,842, issued Sept. 12, 2000. Tirofiban is commercially available as AGGRASTAT® from Merck & Co., Inc., (Whitehouse Station, N.J.), manufactured by Baxter Healthcare Corp. (Deerfield, Ill.) and Ben Venue Labs. (Bedford, Ohio). Tirofiban,has the structure illustrated in Example 10, Structure 2, and has an in vivo circulatory half-life of approximately two hours. The pseudo-antibody is created by attaching an additional moiety to an aromatic site on the molecule, such that the additional moiety (depicted as “X” in Structure 2), is or contains a functional group capable of forming the pseudo-antibody structure, as long as some activity of the parent compound is retained.

Other examples of non-peptide target binding moieties that may be included in the pseudo-antibodies of the present invention include leflunomide (ARAVA™), which has the chemical name α,α,α-Trifluoro-5-methyl-4-isoxazolecarboxy-p-toluidide. Leflunomide is a a prodrug which is changed in the body to an active metabolite. An immuno-suppressive agent, it inhibits pyrimidine synthesis and thus reduces the production of immune cells that attack joints, and may be useful for relief of the signs and symptoms of arthritis.

In another embodiment of the instant invention, the pseudo-antibody construct includes a moiety that inhibits matrix metalloproteases (MMPs). MMPs are involved in invasion, metastasis and angiogenesis. MMPs 2 & 9 are overexpressed in the tumor/stroma of multiple cancers, and are thus attractive targets for inhibition. BAY12-9566 is a selective, non-peptidic biphenyl inhibitor of MMPs (MMPI), exhibiting nM inhibitory activity against MMPs 2, 3 & 9 with anti-invasive, anti-metastatic and anti-angiogenic activity in preclinical models and clinical evaluations in human patients. Lathia et al., Proc. 1999 AACR, NCI, EORTC Int'l Conf., Am. Assoc. Cancer Res. MMPIs, often thought of as promising anti-cancer therapeuticals, are also being investigated for use in rheumatoid arthritis therapy. Other MMPIs include Marimastat and BB-2983. See, e.g, Boasberg et al., 15 Proc. Ann. Meeting Am. Soc. Clin. Oncol. A671 (1996).

The pseudo-antibodies of the present invention also include moieties such as receptors, or fragments thereof, and activated receptors, i.e., peptides associated with their corresponding receptors, or fragments thereof. These complexes may mimic activated receptors and thus affect a particular biological activity. Alternatively, the receptor can be genetically re-engineered to adopt the activated conformation. For example, the thrombin-bound conformation of fibrinopeptide A exhibits a strand-turn-strand motif, with a β-turn centered at residues Glu-11 and Gly-12. Molecular modeling analysis indicates that the published fibrinopeptide conformation cannot bind reasonably to thrombin, but that reorientation of two residues by alignment with bovine pancreatic trypsin inhibitor provides a good fit within the deep thrombin cleft and satisfies all of the experimental nuclear Overhauser effect data. Based on this analysis, a researchers were able to successfully design and synthesize hybrid peptide mimetic substrates and inhibitors that mimic the proposed β-turn structure. The results indicate that the turn conformation is an important aspect of thrombin specificity, and that the turn mimetic design successfully mimics the thrombin-bound conformation of fibrinopeptide. Nakanishi et al., 89(5) PNAS 1705-09 (1992).

Another example of activated-receptor moieties concerns the peptido mimetics of the erythropoietin (Epo) receptor. By way of background, the binding of Epo to the Epo receptor (EpoR) is crucial for production of mature red blood cells. The Epo-bound, activated EpoR is a dimer. See, e.g., Constantinescu et al., 98 PNAS 4379-84 (2001). In its natural state, the first EpoR in the dimer binds Epo with a high affinity whereas the second EpoR molecule binds to the complex with a low affinity. Bivalent anti-EpoR antibodies have been reported to activate EopR, probably by dimerization of the EpoR. Additionally, small synthetic peptides, that do not have any sequence homology with the Epo molecule, are also able to mimic the biologic effects of Epo but with a lower affinity. Their mechanism of action is probably also based on the capacity to produce dimerization of the EpoR. Hence, an embodiment of the present invention provides for a pseudo-antibody comprising an activated EpoR mimetic.

In another preferred embodiment of the invention, the pseudo-antibody may include antimicrobial agents or portions thereof, which include antibacterial agents, antivirals agents, antifungal agents, antimycobacterial agents, and antiparasitic agents. Antibacterials include, but are not limited to, Beta-lactams (such as Penicillins and Cephalosporins), Aminoglycosides (such as Gentamicin), Macrolides (such as Erythromycin), Fluoroquinolones, Metronidazole, Sulfonamides, Tetracyclines, Trimethroprim, and Vancomycin. Antifungal agents include, but are not limited to Amphotericin, Fluconazole, Flucytosine, Itraconazole, and Ketoconazole. Antiparasitic agents include, but are not limited to, Ivermectin, Mebendazole, Mefloquine, Pentamidine, Praziquantel, Pyrimethamine, and Quinine. Antiviral agents include, but are not limited to, Acyclovir, Amantadine, Didanosine, Famciclovir, Foscarnet, Ganciclovir, Rimatandine, Stavudine, Zalcitabine, and Zidovudine. Antimycobacterial agents include, but are not limited to, Isoniazid, Rifampin, Streptomycin, Dapsone. SANFORD ET AL., GUIDE TO ANTIMICROBIAL THERAPY (25th ed., Antimicrobial Therapy, Inc., Dallas, Tex. 1995).

In another embodiment of the invention, the pseudo-antibody targets a cell cycle protein. In yet another embodiment of the invention, the pseudo-antibody includes a cell cycle protein, or a functionally active portion of a cell cycle protein. These cell cycle proteins are known in the art, and include cyclins, such as G₁ cyclins, S-phase cyclins, M-phase cyclins, cyclin A, cyclin D and cyclin E; the cyclin-dependent kinases (CDKs), such as G₁ CDKs, S-phase CDKs and M-phase CDKs, CDK2, CDK4 and CDK 6; and the tumor suppressor genes such as Rb and p53. Cell cycle proteins also include those involved in apoptosis, such as Bc1-2 and caspase proteins; proteins associated with Cdc42 signaling, p70 S6 kinase and PAK regulation; and integrins, discussed elsewhere. Also included in the cell cycle proteins of the present invention are anaphase-promoting complex (APC) and other proteolytic enzymes. The APC triggers the events leading to destruction of the cohesins and thus allowing sister chromatids to separate, and degrades the mitotic (M-phase) cyclins. Other relevant cell cycle proteins include S-phase promoting factor, M-phase promoting factor that activates APC. Kimball, Kimball's Biology Pages, at http://www.ultranet.com/˜jkimball/BiologyPages.

The pseudo-antibody of the present invention may also incorporate or target a particular antigen. Antigens, in a broad sense, may include any molecule to which an antibody, or functional fragment thereof, binds. Such antigens may be pathogen derived, and be associated with either MHC class I or MHC class II reactions. These antigens may be proteinaceous or include carbohydrates, such as polysaccharides, glycoproteins, or lipids. Carbohydrate and lipid antigens are present on cell surfaces of all types of cells, including normal human blood cells and foreign, bacterial cell walls or viral membranes. Nucleic acids may also be antigenic when associated with proteins, and are hence included within the scope of antigens encompassed in the present invention. See SEARS, IMMUNOLOGY (W. H. Freeman & Co. and Sumanas, Inc., 1997), available on-line at http://www.whfreeman.com/immunology.

For example, antigens may be derived from a pathogen, such as a virus, bacterium, mycoplasm, fungus, parasite, or from another foreign substance, such as a toxin. Such bacterial antigens may include or be derived from Bacillus anthracis, Bacillus tetani, Bordetella pertusis; Brucella spp., Corynebacterium diphtheriae, Clostridium botulinum, Clostridium perfringens, Coxiella burnetii, Francisella tularensis, Mycobacterium leprae, Mycobacterium tuberculosis, Salmonella typhimurium, Streptococcus pneumoniae, Escherichia coli, Haemophilus influenzae, Shigella spp., Staphylococcus aureus, Neisseria gonorrhoeae, Neisseria meningitidis, Treponema pallidum, Yersinia pestis, Vibrio cholerae. Often, the oligosaccharide structures of the outer cell walls of these microbes afford superior protective immunity, but must be conjugated to an appropriate carrier for that effect.

Viruses and viral antigens that are within the scope of the current invention include, but are not limited to, HBeAg, Hepatitis B Core, Hepatitis B Surface Antigen, Cytomegalovirus B, HIV-1 gag, HIV-1 nef, HIV-1 env, HIV-1 gp41-1, HIV-1 p24, HIV-1 MN gp120, HIV-2 env, HIV-2 gp 36, HCV Core, HCV NS4, HCV NS3, HCV p22 nucleocapsid, HPV L1 capsid, HSV-1 gD, HSV-1 gG, HSV-2 gG, HSV-II, Influenza A (H1N1), Influenza A (H3N2), Influenza B, Parainfluenza Virus Type 1, Epstein Barr virus capsid antigen, Epstein Barr virus, Poxviridae Variola major, Poxviridae Variola minor, Rotavirus, Rubella virus, Respiratory Syncytial Virus, Surface Antigens of the Syphilis spirochete, Mumps Virus Antigen, Varicella zoster Virus Antigen and Filoviridae.

Other parasitic pathogens such as Chlamydia trachomatis, Plasmodium falciparum, and Toxoplasma gonzdii may also provide antigens for, or be targeted by, the pseudo-antibody of the present invention. Numerous bacterial and viral, and other microbe-generated antigens are available from commercial suppliers such as Research Diagnostics, Inc. (Flanders, N.J.).

Toxins, toxoids, or antigenic portions of either, within the scope of the present invention include those produced by bacteria, such as diphteria toxin, tetanus toxin, botulin toxin and enterotoxin B; those produced by plants, such as Ricin toxin from the castor bean Ricinus cummunis. Mycotoxins, produced by fungi, that may serve in the present invention include diacetoxyscirpenol (DAS), Nivalenol, 4-Deoxynivalenol (DON), and T-2 Toxin. Other toxins and toxoids produced by or derived from other plants, snakes, fish, frogs, spiders, scorpions, blue-green algae, snails may also be incorporated in the pseudo-antibody constructs of the present invention.

A use of antigen constructs can be as immunogens to elicit an immune response in animals for the generation of antibodies or as synthetic vaccines in man to elicit a protective immune response.

Antigens included in the pseudo-antibody constructs of the present invention may also serve as markers for particular cell types, or as targets for an agent interacting with that cell type. Examples include Human Leukocyte Antigens (HLA markers), MHC Class I and Class II, the numerous CD markers useful for identifying T-cells and the physiological states thereof. Alternatively, antigens may serve as “markers” for a particular disease or condition, or as targets of a therapeutic agent. Examples include, Prostate Specific Antigen, Pregnancy specific beta 1 glycoprotein (SP1), Thyroid Microsomal Antigen, and Urine Protein 1. Antigens may include those defined as “self” implicated in autoimmune diseases. Haptens, low molecular weight compounds such as drugs or antibiotics that are too small to cause an immune response unless they are coupled with much larger entities, may serve as antigens when coupled to the pseudo-antibody of the present invention. See ROITT ET AL., IMMUNOLOGY (5th ed., 1998); BENJAMINI ET AL., IMMUNOLOGY, A SHORT COURSE (3rd ed., 1996).

The pseudo-antibody of the present invention may also include an organic moiety to which, through the optional use of a linker, the target-binding moiety is attached. The organic moiety serves to position the target-binding moiety to optimize avidity, affinity, and/or circulating half-life. This moiety can be a hydrophilic polymeric group, a simple or complex carbohydrate, a fatty acid group, a fatty acid ester group, a lipid group, or a phospholipid group. More specifically, polyglycols are hydrophilic polymers that have one or more terminal hydroxy groups, such as polyethylene glycol, polypropylene glycol, polyvinyl pyrrolidone, homo-polyamino acids, hetero-polyamino acids, and polyamides. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

PEG is a generic name for mixtures of condensation polymers of ethylene oxide and water, represented by the general formula H(OCH₂CH₂)ₙ OH, in which n is greater or equal to 4. Those PEGs with an average molecular weight of about 200 to 700 are liquid, and those above 1000 are waxlike solids. PEGs can be esterified with fatty acids to produce non-ionic surfactants in which the PEG functions as the hydrophile. PEGs increase the water solubility of a final product. Higher molecular PEGs impart a greater degree of water solubility than lower molecular weight PEGs.

PPGs are water soluble at low molecular weights (P425), but most PPGs are considered sparingly soluble in water. The secondary hydroxy group of polypropylene glycols is not as reactive as the primary hydroxy group on PEGs.

The pseudo-antibodies of the invention comprise at least one target-binding moiety bound to an organic moiety. In the instance in which the target-binding moiety is an antibody, the organic moiety may be covalently bonded to a carboxyl-terminus of the antibody and/or covalently bonded to the sulfur atom of a cysteinyl residue of the antibody and/or attached by other site-specific methodology such as enzyme-catalyzed transamidation. Thus, the invention provides antibodies comprising site-specific modifications. For example, a modified Fab of an IgG can comprise a PEG moiety, which is bonded to the carboxyl-terminus of the heavy chain. In another embodiment, several modified Fab′ fragments are each bonded to a PEG molecule by sulfur atom of one of the cysteinyl residues that are contained within the hinge region of the heavy chain (the cysteine residues in the hinge region which form inter-chain disulfide bonds in the corresponding IgG or F(ab1). In yet another embodiment, at least two modified Fab fragments, generated through the action of achromopeptidase, are bonded to one PEG moiety at the carboxyl-terminus of the heavy chain.

Attachment of the hydrophilic polymer can be by non-site specific means, under conditions that do not adversely affect the activity of the target-binding moiety, although site-specific attachment is preferred. Examples of methods of attachment include, but are not limited to: (a) Glyoxyl modification of a N-terminal amino group followed by reductive alkylation with an amine, hydrazine, oxime, semicarbazide, or other appropriate nuleophile; (b) Periodic acid oxidation of one or more carbohydrates on a moiety, followed by reductive alkylation with an amine, hydrazine, oxime, semicarbazide, or other nucleophile; (c) Reverse proteolysis to attach an organic moiety containing a nucleophile to the C- or N-termini of a peptide, followed by reductive alkylation, or reaction with a suitable electrophile; and (d) Production of a recombinant peptide containing one or more additional cysteines, followed by its reaction with a suitable maleanide to form a thioether or activated thiol to form a disulfide, or halo compound to form a thioether. Other methods that may be employed are known to those of ordinary skill in the art. See LUNDBLAD, TECHNIQUES IN PROTEIN MODIFICATION (CRC Press, 1995). A specific example of N-terminal derivatization of EPO with an unfunctionalized PEG is discussed in U.S. Pat. No. 6,077,939. See also WO 00/26256, published May11, 2000.

Additionally, in another embodiment of the invention, an additional organic molecule is included in the pseudo-antibody construct. This additional organic molecule is selected from the group consisting of fatty acids, dicarboxylic acids, monoesters or monoamides of dicarboxylic acids, lipids containing saturated fatty acids, lipids containing unsaturated fatty acids, lipids containing mixtures of unsaturated fatty acids, simple carbohydrates, complex carbohydrates, carbocycles (such as steroids), heterocycles (such as alkaloids), amino acid chains, proteins, enzymes, enzyme cofactors, and vitamins. In yet another embodiment of the invention, the additional organic molecule is a lipid. In a yet another preferred embodiment of the invention, this molecule is disteroylphosphatidyl-ethanolamine (DSPE).

As noted previously, the pseudo-antibody of the present invention may affect a specific ligand, such as but not limited to where such pseudo-antibody modulates, decreases, increases, antagonizes, angonizes, mitigates, alleviates, blocks, inhibits, abrogates and/or interferes with at least one biological molecule's activity or binding, or with a receptor activity or binding, in vitro, in situ and/or in vivo. The pseudo-antibodies of the present invention can be used to measure or effect in an cell, tissue, organ or animal (including mammals and humans), to diagnose, monitor, modulate, treat, alleviate, help prevent the incidence of, or reduce the symptoms of, at least one condition. In particular, the pseudo-antibody constructs may be used: to treat stenosis and/or restenosis following a vascular intervention; to prevent ischemia; to inhibit the growth and/or metastasis of a tumor; to inhibit a biological process mediated by the binding of a ligand to either or both of GPIIb/IIIa and αᵥβ3, expressed on the plasma membrane of a cell; and to inhibit angiogenesis. Such a method can comprise administering an effective amount of a composition or a pharmaceutical composition comprising at least one pseudo-antibody to a cell, tissue, organ, animal or patient in need of such modulation, treatment, alleviation, prevention, or reduction in symptoms, effects or mechanisms. The effective amount can comprise an amount of about 0.001 mg/kg to 500 mg/kg per single (e.g., bolus), multiple or continuous administration, or to achieve a serum concentration of 0.01-5000 μg/ml serum concentration per single, multiple, or continuous administration, or any effective range or value therein, as done and determined using known methods, as described herein or known in the relevant arts.

### EXAMPLES

Certain constructs described herein may be similar to previously disclosed compounds, such as a Fab′ antibody fragment with two PEG chains. WO 0026256; published May 11, 2000. The descriptions herein are not meant to be exclusive of all previously disclosed compounds but are meant to define the broadest scope of this concept.

For purposes of illustrating the scope of the invention, a Fab molecule is used in pseudo-antibody (Ψ Ab) constructs. The use of this example is not meant to limit the scope of the invention to antibody fragments. The Fab contains a single free thiol (an SH group) in the form of a cysteine, located toward or on the C-terminus of the heavy or light chain. By analogy, a single chain antibody, peptide, or organic molecule with a free thiol could also be used. While the method of constructing the example Ψ Abs uses the spontaneous reaction of a thiol with a maleimide, other methods of covalent bond formation are envisioned as well. Examples, not meant to limit or define the scope of the invention disclosure, include the spontaneous reaction of azides with trivalent phosphorus species such as dimethoxy-alkylphosphites to form phosphoramidates, the reductive alkylation of carbonyl compounds with amine derivatives and the spontaneous reaction of thiols with bromoacetyl derivatives to form thioethers.

### Example 1

Construct 1, shown in scheme 1, illustrates the addition of a single Fab to a maleimido-PEG, where the molecular weight of the PEG is such that the construct has a longer in vivo half-life than Fab₁, R can be an alkoxy group such as methoxyl or a compound selected from the structural categories of carbohydrates, saturated or unsaturated mono- or di-carboxylic acids, monoesters or amides of saturated or unsaturated di-carboxylic acids, higher alkoxy groups, lipids or other biologically compatible organic molecules. X₁ is an optional linker or spacer between the maleimide moiety and the PEG. The preferred method of synthesis for these constructs is shown in Scheme 1, where the R group has been previously attached to the PEG; however, synthetic schemes can be envisioned where the R group is attached to the PEG after the Fab-maleimide reaction. Additional activity can be imparted to these constructs by the R group.

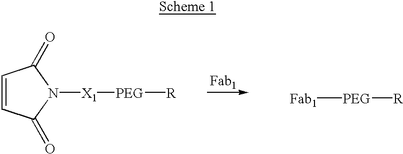

### Example 2

Construct 2, shown in Scheme 2, has identical Fabs on opposite ends of a PEG where the molecular weight of the PEG is such that the construct has a longer in vivo half-life than Fab₁. X₁ and X₂ are linkers between the PEG and the maleimide groups and may be either structurally identical or structurally unique. This type of construct has the advantage over an IgG in that the two Fabs can bind to identical receptors that are significantly further apart than could be bridged by a conventional immnunoglobulin.

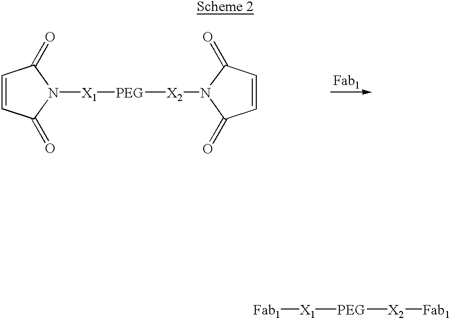

### Example 3

Construct 3, shown in Scheme 3, is composed of different Fabs on opposite ends of a PEG where the molecular weight of the PEG is such that the construct has a longer in vivo half-life than the Fabs from which it is constructed. This type of bifunctional Ψ Ab construct has the advantage over a conventional bifunctional antibody fragment in that the two Fabs can bind to non-identical receptors that are significantly further apart than could be bridged by a conventional bifunctional construct. The synthesis of this type of construct is illustrated using sequential addition of the Fabs to a bis-maleimido-PEG, although other synthetic routes can be envisioned as well. This type of construct is well suited to a synthetic route in which the chemistry of attachment of the two Fabs is different, or the addition of one maleimide to the PEG is done after the addition of the first Fab.

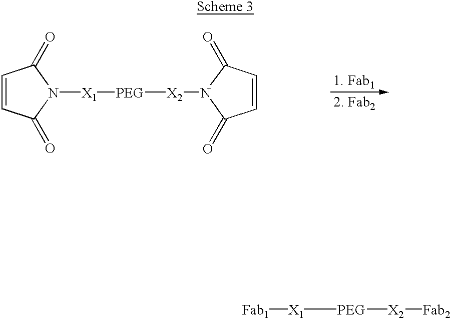

### Example 4

Construct 4, shown in Scheme 4, has two identical Fabs on the same end of a PEG, where Q can be an alkoxy group such as methoxyl or a compound selected from the structural categories of carbohydrates, saturated or unsaturated mono- or di-carboxylic acids, monoesters or amides of saturated or unsaturated di-carboxylic acids, higher alkoxy groups, lipids or other biologically compatible organic molecules. When the Fab moiety has a single free —SH group, maleimide is used. In a preferred embodiment, Q is diesteroylphosphatidylethanolamine. Q can be also be an active molecule such as a toxin or a radioisotope, or a marker such as GFP. Y₁ and Z₁ are linkers or spacers between the maleimide moiety and the PEG and can be the same or different. W is a trifunctional moiety such that one functionality can be attached to a PEG and the other two can be attached to the linkers Y₁ and Z₁. As an example, Q is methoxyl, PEG is NH₂-PEG, W₁ is Lysine, and Y₁ and Z₁ are both propionyl.

In this and further examples, when the target binding moiety has an aldehyde or ketone functionality and the organic moiety contains a hydrazine functionality, then reductive alkylation may be used to form a covalent C—N bond. Another possibility is the reverse, where the target binding moiety contains a hydrazine functionality and the organic moiety contains an aldehyde or ketone, then reductive alkylation also leads to the formation of a covalent C—N bond. Alternatively, the target binding moiety can contain a single free —SH group and the organic moiety contains a bromoacetyl moiety, in which case, these groups spontaneously react (under appropriate pH control) to form a thioether bond. If, for example, the target binding moiety contains a hydrazine and the organic moiety contains a 1,3-di-carbonyl moiety or a 1,4-dicarbonyl moiety, then reaction of these functionalities would lead to stable 5- or 6-membered heterocyclic systems. The reverse configuration would also work: The target binding moiety could contain an azide and the organic moiety could contain a trivalent phosphorus moiety, giving spontaneous reaction for form a covalent phosphoramidate bond.

This type of bifunctional Ψ Ab construct has the advantage over a conventional Fab′₂ antibody fragment in that incorporation of the PEG can increase the molecular size of the construct to IgG size without the associated Fc activity.

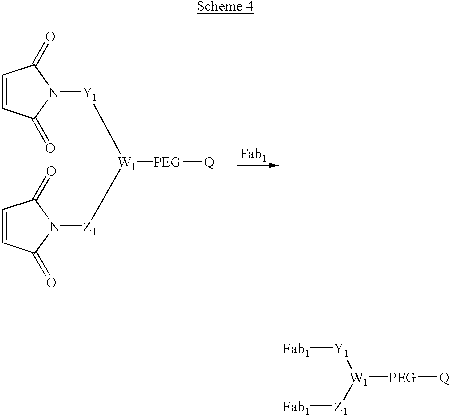

### Example 5

Construct 5, shown in Scheme 5, has two different Fabs on the same end of a PEG, where Q can be an alkoxy group such as methoxyl or a compound selected from the structural categories of carbohydrates, saturated or unsaturated mono- or di-carboxylic acids, monoesters or amides of saturated or unsaturated di-carboxylic acids, higher alkoxy groups, lipids or other biologically compatible organic molecules. Y₁ and Z₁ are linkers or spacers between the maleimide moiety and the PEG, and can be the same or different. W is a trifunctional moiety such that one functionality can be attached to a PEG and the other two can be attached to the linkers Y₁ and Z₁. As an example, Q is methoxyl, PEG is NH₂-PEG, W₁ is Lysine and Y₁ and Z₁ are both propionyl. The synthesis of this type of construct is illustrated using sequential addition of the Fabs to a bis-maleimido-PEG, although other synthetic routes can be envisioned as well. This type of construct is well suited to a synthetic route in which the chemistry of attachment of the two Fabs is different, or the addition of one maleimide to the PEG is done after the addition of the first Fab. This type of bifunctional Ψ Ab construct has the advantage over a conventional bifunctional antibody fragment in that incorporation of the PEG can increase the molecular size of the construct to IgG size without the associated Fc activity and additional activity can be imparted to these constructs by the Q group.

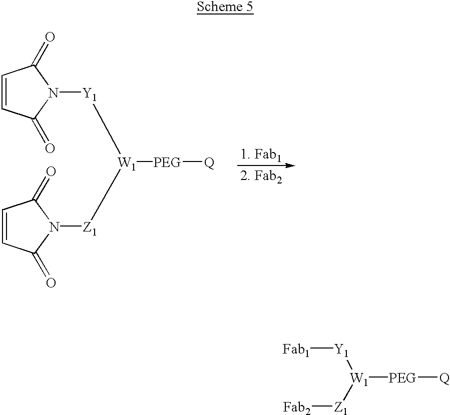

### Example 6

Construct 6, shown in Scheme 6, has two different Fabs on each end of a PEG. Y₁, Y₂, Z₁ and Z2 are linkers or spacers between the maleimide moiety and the PEG and can be the same

or different. W₁ and W₂ are trifunctional moieties such that one functionality can be attached to a PEG and the other two can be attached to the linkers Y₁, Y₂, Z₁ and Z₂. As an example, PEG is NH₂-PEG, W₁ and W₂ are Lysine and Y₁, Y₂, Z₁ and Z₂ are propionyl. The synthesis of this type of construct is illustrated using addition of the Fabs to a bis-maleimido-PEG, although other synthetic routes can be envisioned as well. This type of tetravalent Ψ Ab construct has the advantage over a conventional antibody fragment in that incorporation of the PEG can increase the molecular size of the construct to IgG size without the associated Fc activity and the multiple binding capacity can increase avidity.

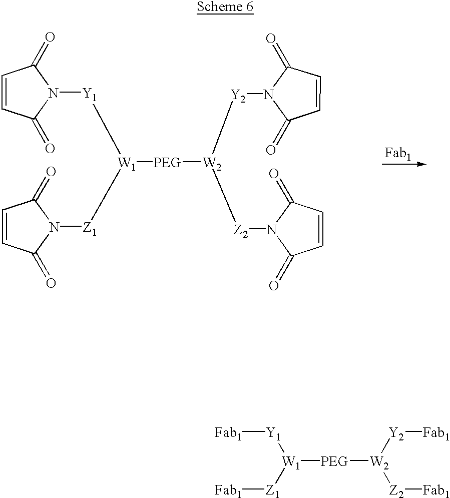

### Example 7

Construct 7, shown in Scheme 7, has two different sets of Fabs on opposite ends of a PEG. Y₁, Y₂, Z₁ and Z₂ are linkers or spacers between the maleimide moiety and the PEG and can be the same or different. W₁ and W₂ are trifunctional moieties such that one functionality can be attached to a PEG and the other two can be attached to the linkers Y₁, Y₂, Z₁ and Z₂. As an example, PEG is NH₂-PEG-NH₂, W₁ and W₂ are Lysine and Y₁, Y₂, Z₁ and Z₂ are propionyl.

The synthesis of this type of construct is illustrated using sequential addition of the Fabs to a bis-maleimido-PEG in, although other synthetic routes can be envisioned as well. This type of tetravalent Ψ Ab construct has the advantage over a conventional antibody fragment in that incorporation of the PEG can increase the molecular size of the construct to IgG size without the associated Fc activity and the multiple binding capacity can increase avidity. Schemes 8 and 9 show two routes to these constructs, although other routes can be envisioned as well. L and M are groups that will react with groups at the ends of the PEG. For example L may be an active ester when the PEG moiety terminates in an amino group and would lead to the formation of an amide linkage or they may be hydrazides when the PEG moiety terminates in an aldehyde function and would lead to a hydrazide by way of reductive alklyation. Other groups may be envisioned as well. L and M may be identical or different depending on the specific assembly strategy. This type of bis-Ψ Ab construct has the advantage of being able to target two different antigens with IgG avidity in a single molecule.

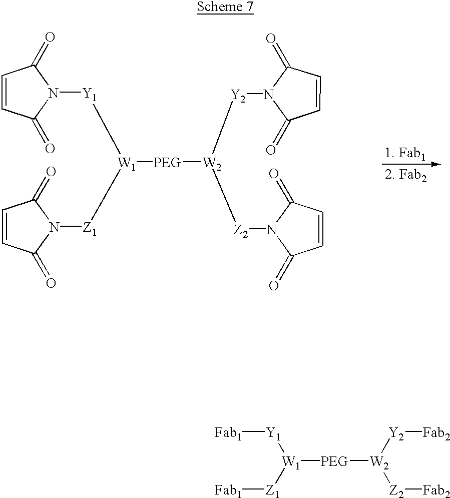

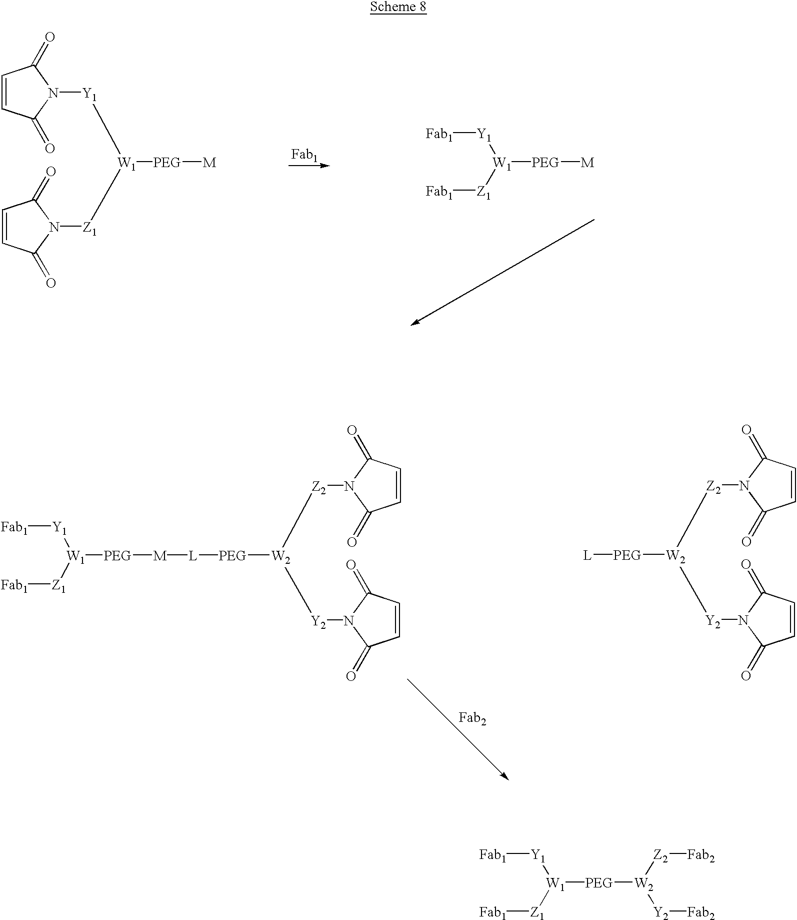

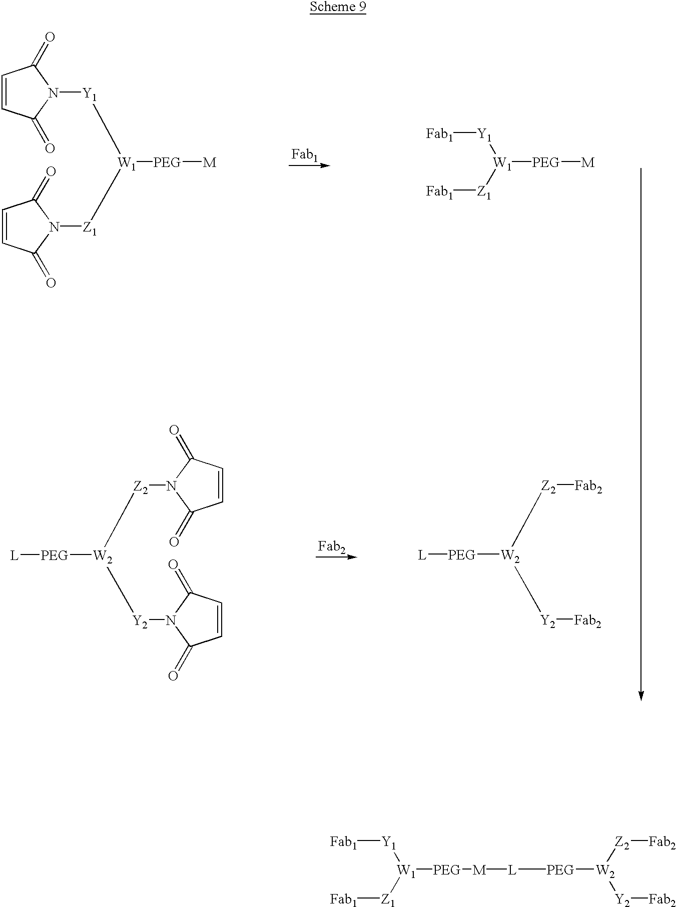

### Example 8

Construct 8, shown in Scheme 10, has three identical Fabs on the same end of a PEG where S can be H, an alkoxy group such as methoxyl or a compound selected from the structural categories of carbohydrates, saturated or unsaturated mono- or di-carboxylic acids, monoesters or amides of saturated or unsaturated di-carboxylic acids, higher alkoxy groups, lipids or other biologically compatible organic molecules. X₁, X₂ and X₃ are linkers or spacers between the maleimide moiety and the PEG and can be the same or different. Y is a trifunctional moiety such that one functionality can be attached to a PEG and the other two can be attached to the linkers X₁, X₂ and X₃. As an example, S is methoxyl, PEG is NH₂-PEG, Y is Lysyl-Lysine and X₁, X₂ and X₃ are propionyl.

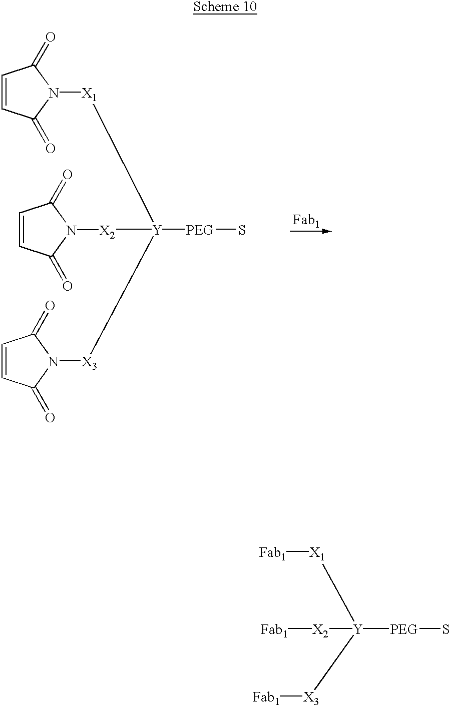

In addition, one can readily envision higher order constructs with different numbers of identical or different Fabs attached to the ends of linear or branched PEGs or more complex structures involving multifunctional PEGs (e.g., NH₂-PEG₁-NH-PEG₂-NH₂).

### Example 9

Examples of the types of structures that can be used as target binding moieties are REOPRO®-TC Fabs, where REOPRO® Fab is derived from the antibody c7E3 and TC represents the addition of threonyl-cysteine to the C-terminus of the heavy chain and the compound shown in Structure 1, capable of inhibiting platelet aggregation by binding to the GPIIb/IIIa receptor. Cysteines can be incorporated into other positions in a Fab as well. It need not be on the C-terminus. In this example, X is or contains a functional group capable of forming the Ψ Ab structure. Alternatively, X is hydrogen, and the carboxylic acid of cysteine forms an amide with an amino group that is attached to the organic moiety. Then, instead of NH₂—, as shown, it would be R—NH. The position of X is selected at any of those sites on the molecule at which substitution allows the parent structure to retain some activity.

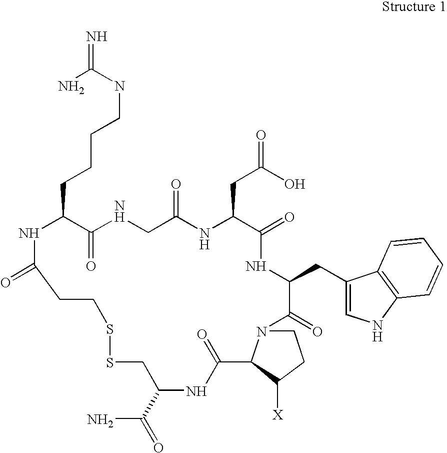

### Example 10

Another example of a structure that can be used for a target binding moiety is shown in Structure 2, a compound capable of inhibiting platelet aggregation by binding to the GPIIb/IIIa receptor, where X is or contains a functional group capable of forming the Ψ Ab structure. The position of X is selected at any of those aromatic sites on the molecule for which substitution will retain some activity of the parent structure, and is not limited to that position depicted in the drawing.

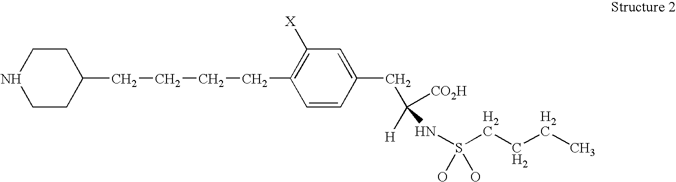

### Example 11

Another example of a structure that can be used for a Fab is the peptide shown in Structure 3, a compound capable of binding to the erythropoietin receptor and stimulating erythropoiesis, where X is or contains a functional group capable of forming the Ψ Ab structure. One specific example is where X is an aldehyde containing moiety; however, other functional groups could be inserted as well. In the case where a cysteine is to be used to form the Ψ Ab structure, amino acids in the parent peptide could be substituted as well if they will not eliminate the activity of the parent structure. Preferably, attachment is at the amino- or carboxy-terminus of the molecule.

### XGGTYS-cyclo(CHFGPLTWVC)—KPQGG

### Structure 3

### Example 12.

This example provides for a pseudo-antibody with the structure A-(PEG-Q)ₙ; wherein A is a Fab fragment, and Q is a fatty acid or lipid, and n is 1 or 2. Interstingly, the Fab-PEG-Q pseudo-antibody may have a greater circulating half-life compared to its counterpart Fab-PEG pseudo-antibody. In this example, Q is either diesteroylphosphatidyl-ethanolamine (DSPE) or palmatoyl (PAL). These pseudo-antibodies may be considered superior to unmodified Fabs, in that antigen-binding is retained while circulating half-life increases. Indeed, the increased circulating half-life may be advantageous even if antigen-binding activity is decreased by the addition of the organic moiety.

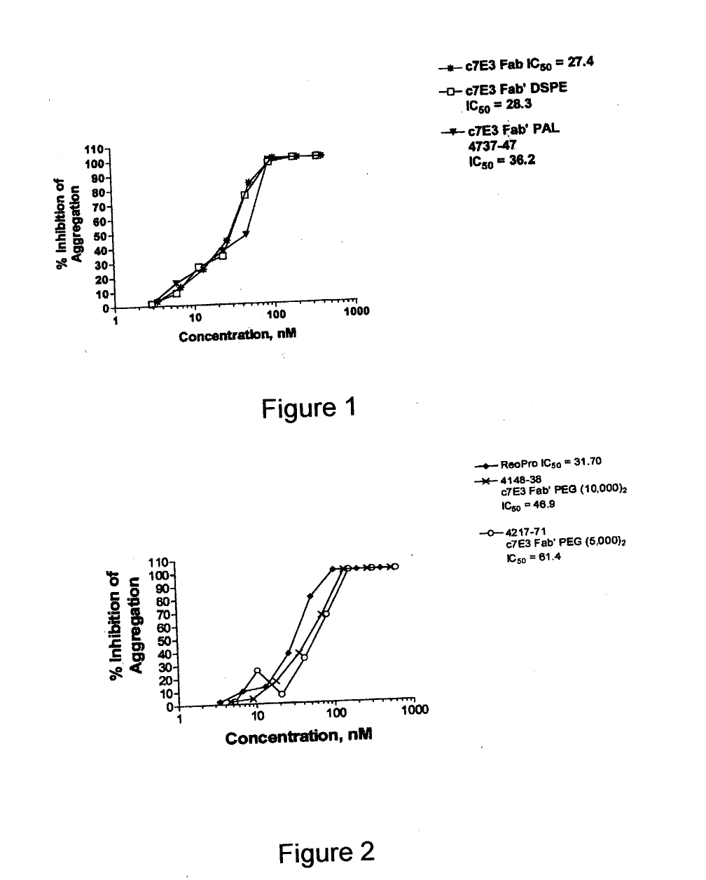

The organic moieties portions of these constructs may also be dimerized, such that n=2. For example, the antibody fragment 7E3 Fab′ was used to construct the pseudo-antibody 7E3 Fab′ (PEG_{3.4k}-DSPE)₂ and the pseudo-antibody 7E3 Fab′ (PEG_{3.4.k}-PAL)₂ and the in vitro activities were compared with unmodified 7E3 Fab′. The activities of pseudo-antibodies and the unmodified Fab were similar, as indicated in FIG. 1.

Additionally, 7E3 Fab′ was used to construct the pseudo-antibodies 7E3 Fab′ (PEG₅ₖ)₂ and 7E3 Fab′ (PEG₁₀ₖ)₂ and the in vitro activites were compared with the unmodified antibody fragment ReoPro®. These constructs exhibited somewhat lower in vitro activity than the unmodified antibody fragment, yet binding activity was clearly retained, as indicated in FIG. 2.

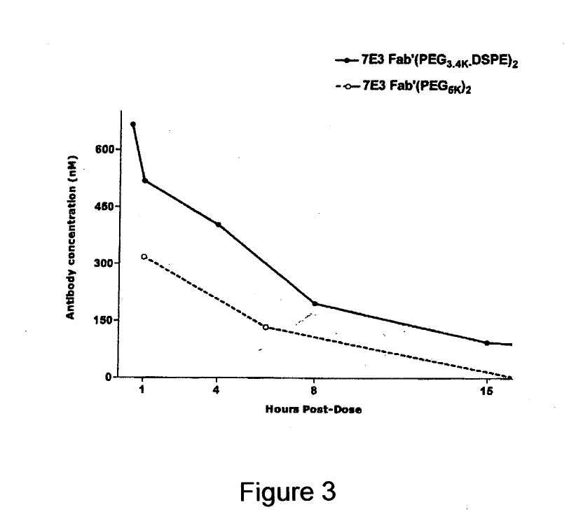

For in vivo pharmacokinetic analysis, c7E3 Fab′ (PEG_{3.4k}-DSPE)2 and c7E3 Fab′ (PEG₅ₖ) were prepared, and given to mice in equimolar doses. The results are depicted in FIG. 3. Although the c7E3 Fab′ (PEG₅ₖ) pseudo-antibody has a higher molecular weight and is larger than the c7E3 Fab′ (PEG_{3.4k}-DSPE)₂ pseudo-antibody, it was cleared faster. The slower rate of clearance of the c7E3 Fab′ (PEG_{3.4k}-DSPE)₂ pseudo-antibody construct may be contributed to the incorporation of the lipid moiety in the pseudo-antibody construct.

Other structures can be envisioned as well. Preferred structures are those that bind to a biological molecule to block binding to another biological molecule or bind to a biological molecule to initiate a biological event.

I claim:

1. A pseudo-antibody comprising an organic moiety covalently coupled to three or more identical target-binding moieties, wherein said target-binding moieties are selected from the group consisting of a protein, a peptide, a peptidomimetic, and a non-peptide molecule that binds to a specific targeted biological molecule.

2. The pseudo-antibody of claim 1, wherein said pseudo-antibody exhibits increased avidity compared to the unmodified target-binding moiety from which it is derived.

3. The pseudo-antibody of claim 1, wherein said organic moiety is selected from the group consisting of a hydrophilic polymeric group, a fatty acid group, a fatty acid ester group, a simple carbohydrate, a complex carbohydrate, a lipid, and a phospholipid.

4. The pseudo-antibody of claim 3, wherein said organic moiety is a hydrophilic polymeric group.

5. The pseudo-antibody of claim 4, wherein said hydrophilic polymeric group is present on a polyethylene glycol (PEG) molecule.

6. The pseudo-antibody of claim 5, wherein said PEG molecule of sufficient size to extend the in vivo half-life of an unmodifed target-binding moiety.

7. The pseudo-antibody of claim 1, wherein said target-binding moiety inhibits binding of fibrinogen to GPIIb/IIIa.

8. The pseudo-antibody of claim 1, wherein said target-binding moiety is a protein selected from the group consisting of an antibody, a cytokine, a growth factor, a cell cycle protein, a blood protein, an integrin, a receptor, a neurotransmitter, an antigen, an anti-microbial agent, and any functional or structural equivalent of any of the foregoing.

9. The pseudo-antibody of claim 1, wherein said target-binding moiety is a protein that is a receptor or a functional portion of a receptor for a molecule selected from the group consisting of an antibody, a cytokine, a growth factor, a cell cycle protein, a blood protein, an integrin, a neurotransmitter, an antigen, an anti-microbial agent, and any functional or structural equivalent of any of the foregoing.

10. The pseudo-antibody of claims 8, wherein said target-binding moiety is a Fab.

11. The pseudo-antibody of claim 10, wherein the binding of said Fab to GPIIb/IIIa is competitively inhibited by 7E3.

12. The pseudo-antibody of claim 11, wherein said Fab is selected from the group consisting of 7E3, antigen-binding fragments of 7E3, chimerized 7E3, antigen-binding fragments of chimeric 7E3, humanized 7E3, and antigen-binding fragments of humanized 7E3.

13. The pseudo-antibody of claim 11, wherein said Fab has an increased in vivo serum half-life, compared to an unmodified antibody or unmodified Fab that is competitively inhibited by 7E3.

14. The pseudo-antibody of claim 4,wherein said hydrophilic polymeric group is selected from the group consisting of, linear or branched polyalkane glycol chains, carbohydrate chains, amino acid chains and polyvinyl pyrolidone chains; wherein said hydrophilic polymeric group has a molecular weight of about 800 Daltons to about 120,000 Daltons.

15. The pseudo-antibody of claim 14, wherein said hydrophilic polymeric group is a linear or branched polyalkane glycol chain with a molecular weight greater than about 2,000 Daltons.

16. A pseudo-antibody comprising an organic moiety covalenty coupled to two or more different target-binding moieties, wherein said target-binding moieties are selected from the group consisting of a protein, a peptide, a peptidomimetic, and a non-peptide molecule that binds to a specific targeted biological molecule.

17. The pseudo-antibody of claim 16, wherein said pseudo-antibody exhibits increased avidity compared to the unmodified target-binding moiety from which it is derived.

18. The pseudo-antibody of claim 16 wherein said organic moiety is selected from the group consisting of a hydrophilic polymeric group, a fatty acid group, a fatty acid ester group, a simple carbohydrate, a complex carbohydrate, a lipid, and a phospholipid.

19. The pseudo-antibody of claim 18, wherein said organic moiety is a hydrophilic polymeric group.

20. The pseudo-antibody of claim 19, wherein said hydrophilic polymeric group is present on a polyethylene glycol (PEG) molecule.

21. The pseudo-antibody of claim 20, wherein said PEG molecule of sufficient size to extend the in vivo half life of said unmodifed target-binding moiety.

22. The pseudo-antibody of claim 16, wherein said target-binding moiety inhibits binding of fibrinogen to GPIIb/IIIa.

23. The pseudo-antibody of claim 16, wherein said target-binding moiety is a protein selected from the group consisting of an antibody, a cytokine, a growth factor, a cell cycle protein, a blood protein, an integrin, a receptor, a neurotransmitter, an antigen, an anti-microbial agent, and any functional or structural equivalent of any of the foregoing.

24. The pseudo-antibody of claim 16, wherein said target-binding moiety is a protein that is a receptor or a functional portion of a receptor for a molecule selected from the group consisting of an antibody, a cytokine, a growth factor, a cell cycle protein, a blood protein, an integrin, a neurotransmitter, an antigen, an anti-microbial agent, and any functional or structural equivalent of any of the foregoing.

25. The pseudo-antibody of claims 23, wherein said target-binding moiety is a Fab.

26. The pseudo-antibody of claim 25, wherein the binding of said Fab to GPIIb/IIIa is competitively inhibited by 7E3.

27. The pseudo-antibody of claim 26, wherein said Fab is selected from the group consisting of 7E3, antigen-binding fragments of 7E3, chimeric 7E3, an antigen-binding fragment of chimeric 7E3, humanized 7E3, and antigen-binding fragments of humanized 7E3.

28. The pseudo-antibody of claim 26, wherein said Fab has an increased in vivo serum half-life, compared to an unmodified antibody or unmodified Fab that is competitively inhibited by 7E3.

29. The pseudo-antibody of claim 18,wherein said hydrophilic polymeric group is selected from the group consisting of, linear or branched polyalkane glycol chains, carbohydrate chains, amino acid chains and polyvinyl pyrolidone chains; wherein said hydrophilic polymeric group has a molecular weight of about 800 Daltons to about 120,000 Daltons.

30. The pseudo-antibody of claim 29, wherein said hydrophilic polymeric group is a linear or branched polyalkane glycol chain with a molecular weight greater than about 2,000 Daltons.

31. A pharmaceutical composition comprising a multivalent pseudo-antibody comprising two or more target-binding moieties covalently coupled to a functional molecule.

32. The pharmaceutical composition of claim 31, wherein said functional molecule is a GIIb/IIIa antagonist.

33. The pharmaceutical composition of claim 31, wherein said target-binding moiety is a GIIb/IIIa antagonist.

34. The pharmaceutical composition of claim 32, wherein said pseudo-antibody comprises the following structure: 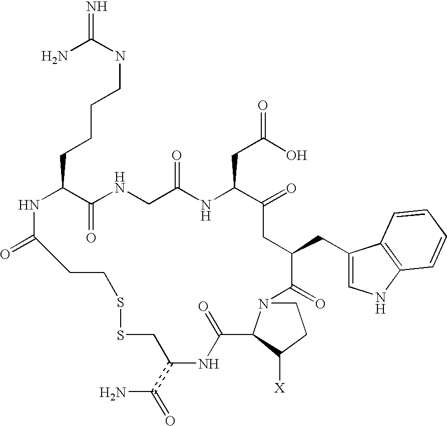
wherein X is or contains a functional group capable of forming the pseudo-antibody structure.

35. The pharmaceutical composition of claim 31, wherein said pseudo-antibody comprises the following structure: 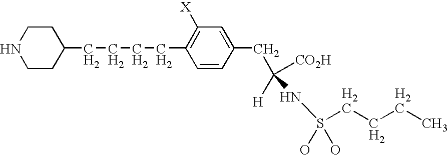
wherein X is or contains a functional group capable of forming the pseudo-antibody structure.

36. A pharmaceutical composition comprising a dimerized peptidomimetic that exhibits enhanced binding to an EPO receptor as compared to its monomered peptidomimetic.

37. The pharmaceutical composition of claim 36, wherein the dimerized peptidomimetic has the structure:
XGGTYS-cyclo(CHFGPLTWVC)—KPQGG
wherein X is hydrazine.

38. The pseudo-antibody of claim 1, further comprising a linker molecule between said antigen-binding-fragment and said organic moiety.

39. The pseudo antibody of claim 16, further comprising a linker molecule between said antigen-binding-fragment and said organic moiety.

40. The pseudo-antibody of claim 1, further comprising an additional functional molecule.

41. The pseudo-antibody of claim 16, further comprising an additional functional molecule.

42. A pseudo-antibody comprising the structure A₁-X₁-PEG-X₂-A₂, wherein A₁ and A₂ are different target-binding moieties each selected from the group consisting of a protein, a peptide, a peptidomimetic, and a non-peptide molecule that binds to a specific targeted biological molecule, wherein X₁ and X₂ are optional linkers between the PEG and the A moieties.

43. The pseudo-antibody of claim 42, wherein said linkers are structurally identical.

44. The pseudo-antibody of claim 42, wherein said linkers structurally unique.

45. The pseudo-antibody of claim 42, wherein said either or both of A₁ or A₂ is a Fab.

46. A pseudo-antibody having the following structure: 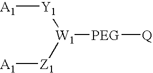
wherein A₁ is selected from the group consisting of a protein, a peptide, a peptidomimetic, and a non-peptide molecule that binds to a specific targeted biological molecule;
wherein Q can be an alkoxy group, such as methoxyl, or a compound selected from the group of structural categories consisting of a carbohydrate, a saturated or unsaturated mono- or di-carboxylic acid, a monoester or amide of a saturated or unsaturated di-carboxylic acid, a higher alkoxy group, a lipid, or other biologically compatible organic molecule;
wherein Y₁ and Z₁ are linkers or spacers between the maleimide moiety and the PEG and can be the same or different; and
wherein W₁ is a trifunctional moiety such that one functionality can be attached to a PEG and the other two can be attached to the linkers Y₁ and Z₁ or directly to A₁ and A₂.

47. The pseudo-antibody of claim 46, in which Q is methoxyl, PEG is NH₂-PEG, W₁ is Lysine, and Y₁ and Z₁ are both propionyl.

48. A pseudo-antibody having the following structure: 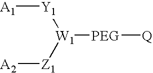
wherein A₁ and A₂ are selected from the group consisting of a protein, a peptide, a peptidomimetic, and a non-peptide molecule that binds to a specific targeted biological molecule, with the proviso that A₁ and A₂ are not identical;
wherein Q can be an alkoxy group, such as methoxyl, or a compound selected from the group of structural categories consisting of a carbohydrate, a saturated or unsaturated mono- or di-carboxylic acid, a monoester or amide of a saturated or unsaturated di-carboxylic acid, a higher alkoxy group, a lipid, or other biologically compatible organic molecule;
wherein Y₁ and Z₁ are optional linkers or spacers between the maleimide moiety and the PEG; and
wherein W₁ is a trifunctional moiety such that one functionality can be attached to a PEG and the other two can be attached either to the linkers Y₁ and Z₁, or directly to A₁ and A₂.

49. The pseudo-antibody of claim 48, wherein Q is methoxyl, PEG is NH₂-PEG, W₁ is Lysine and Y₁ and Z₁ are both propionyl.

50. A pseudo-antibody comprising the following structure: 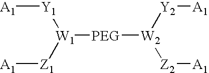
wherein A₁ is selected from the group consisting of a protein, a peptide, a peptidomimetic, and a non-peptide molecule that binds to a specific targeted biological molecule;
wherein Y₁, Y₂, Z₁ and Z₂ are optional linkers or spacers between the maleimide moiety and the PEG; and
wherein W₁ and W₂ are trifunctional moieties such that one functionality can be attached to a PEG and the other two can be attached either to the linkers Y₁, Y₂, Z₁ and Z_{2,} or directly to the A₁ moiety.

51. The pseudo-antibody of claim 50, wherein PEG is NH₂-PEG, W₁ and W₂ are Lysine and Y₁, Y₂, Z₁ and Z₂ are propionyl.

52. A pseudo-antibody comprising the following structure: 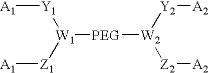
wherein A₁ and A₂ are selected from the group consisting of a protein, a peptide, a peptidomimetic, and a non-peptide molecule that binds to a specific targeted biological molecule, with the proviso that A₁ and A₂ are not identical;
wherein Y₁, Y₂, Z₁ and Z₂ are optional linkers or spacers between the maleimide moiety and the PEG and can be the same or different; and
wherein W₁ and W₂ are trifunctional moieties such that one functionality can be attached to a PEG and the other two can be attached either to the linkers Y₁, Y₂, Z₁ and Z₂, or directly to the A₁ moiety.

53. The pseudo-antibody of claim 52, wherein PEG is NH₂-PEG-NH₂, W₁ and W₂ are Lysine and Y₁, Y₂, Z₁ and Z₂ are propionyl.

54. A pseudo-antibody comprising the following structure: 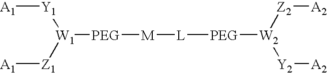
wherein A₁ and A₂ may be identical or different, each selected from the group consisting of a protein, a peptide, a peptidomimetic, and a non-peptide molecule that binds to a specific targeted biological molecule;
wherein Y₁, Y₂, Z₁ and Z₂ are optional linkers or spacers between the maleimide moiety and the PEG and can be the same or different;
wherein W₁ and W₂ are trifunctional moieties such that one functionality can be attached to a PEG and the other two can be attached either to the linkers Y₁, Y₂, Z₁ and Z₂, or directly to the A₁ and A₂ fragments; and
wherein M and L are identical or different, each selected from the group consisting of an amide, an ester, a thioamide, a thioester, a disulfide, and another covalent bond formed by two individual, compatible functional groups.

55. A pseudo-antibody comprising the following structure: 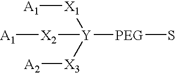
wherein A₁ and A₂ are selected from the group consisting of a protein, a peptide, a peptidomimetic, and a non-peptide molecule that binds to a specific targeted biological molecule, with the proviso that A₁ and A₂ are not identical;
wherein S is a hydrogen, an alkoxy group, such as methoxyl, or a compound selected from the structural categories consisting of a carbohydrate, a saturated or unsaturated mono- or di-carboxylic acid, a monoester or amide of a saturated or unsaturated di-carboxylic acid, a higher alkoxy group, a lipid, and an other biologically compatible organic molecules;
wherein X₁, X₂ and X₃ are linkers or spacers between the maleimide moiety and the PEG and can be the same or different; and
wherein Y is a multifunctional moiety such that one functionality can be attached to a PEG and the other three can be attached to the linkers X₁, X₂ and X₃.

56. The pseudo-antibody comprising the following structure: 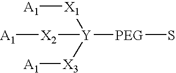
wherein A₁ is selected from the group consisting of a protein, a peptide, a peptidomimetic, and a non-peptide molecule that binds to a specific targeted biological molecule; S is methoxyl; PEG is NH₂-PEG; Y is Lysyl-Lysine; and X₁, X₂ and X₃ are propionyl.

57. A pseudo-antibody comprising the following structure: A₁-(PEG-Q)ₙ; wherein A₁ is selected from the group consisting of a protein, a peptide, a peptidomimetic, and a non-peptide molecule that binds to a specific targeted biological molecule; Q is selected from the group consisting of a fatty acid and a lipid; n is 1 or more, and wherein said A₁-(PEG-Q)ₙ pseudo-antibody has a greater circulating half-life compared to its counterpart A₁-(PEG)ₙ.

58. The pseudo-antibody of claim 57, in which Q is diesteroylphosphatidylethanolamine.

59. The pseudo-antibody of claim 57, in which Q is palmatoyl.

60. A molecule that binds to a primary biological molecule, having at least one or more of the following characteristics selected from the groups consisting of:
multivalent structure with enhanced avidity;
increased molecular size with extended circulating half-life;
specific binding to multiple compounds by a single molecule; and
incorporation of carriers such as lipids, fatty acids, carbohydrates and steroids, that can bind to molecules other than the primary biological molecules and affect distribution to specific locations.

61. A method of inhibiting stenosis and/or restenosis following a vascular intervention procedure in a human comprising administering to said human an effective amount of a composition comprising the pseudo-antibody of claim 1 or claim 16.

62. A method of preventing ischemia in a human comprising administering to said human an effective amount of the pseudo-antibody of claim 1 or claim 16.

63. A method of inhibiting the growth and/or metastasis of a tumor in a human comprising administering to said human an effective amount of the pseudo-antibody of claim 1 or claim 16.

64. A method of inhibiting a process mediated by the binding of a ligand to one of the group consisting of GPIIb/IIIa, αᵥβ₃ and both GPIIb/IIIa, αᵥβ, expressed on the plasma membrane of a cell in a human, comprising administering to said human an effective amount of the pseudo-antibody of claim 1 or claim 16.

65. A method of inhibiting angiogenesis in a human comprising administering to said human an effective amount of the pseudo-antibody of claim 1 or claim 16.

66. The pharmaceutical composition of claim 36, wherein the dimerized peptidomimetic has the structure:
GGTYS-cyclo(CHFGPLTWVC)—KPQGG-R
wherein R is an organic moiety, and the linkage between the carboxylid acid of glycine and R is an amide bond.

67. The pharmaceutical composition of claim 31, wherein said pseudo-antibody comprises the following structure, wherein X is or contains a functional group capable of forming the pseudo-antibody: [chemical structure]
